(19) United States
(12) Patent Application Publication
(10) Pub. No.: US 2011/0237622 A1
(43) Pub. Date: Sep. 29, 2011

(54) RENIN INHIBITORS

(75) Inventors: Austin Chih-Yu Chen, San Diego, CA (US); Daniel Dube, St. Lazare (CA); Pierre-Andre Fournier, Laval (CA); Erich L. Grimm, Baie d'Urfe (CA); Patrick Lacombe, Montreal (CA); Sebastien Laliberte, St. Lazare (CA); Dwight Macdonald, L'Ile Bizard (CA); D. Bruce Mackay, Dollard-des-Ormeaux (CA); Daniel James Mckay, Lexington, MA (US); Tom Yao-Hsiang Wu, San Diego, CA (US)

(73) Assignee: Merck Frosst Canada LTD., Kirkland (CA)

(21) Appl. No.: 13/139,012

(22) PCT Filed: Dec. 8, 2009
(86) PCT No.: PCT/CA2009/001758

§ 371 Date: Jun. 10, 2011

Related U.S. Application Data
(60) Provisional application No. 61/201,367, filed on Dec. 10, 2008.

(51) Int. Cl. A61K 31/4545; C07D 401/04; A61P 9/12; A61P 9/00; A61P 9/10; A61P 13/12; A61P 11/00; A61P 25/22; A61P 25/00; A61P 15/10; A61P 27/06

(52) U.S. Cl. 514/318; 546/194

(57) ABSTRACT

The present invention relates to 3,4-substituted piperidinyl—based renin inhibitor compounds bearing at 4-position oxopyridine or Isoquinolone and having the formula (I): wherein S is Formula (IIa) or Formula (IIb). The invention further relates to pharmaceutical compositions containing said compounds, as well as their use in treating cardiovascular events and renal insufficiency.

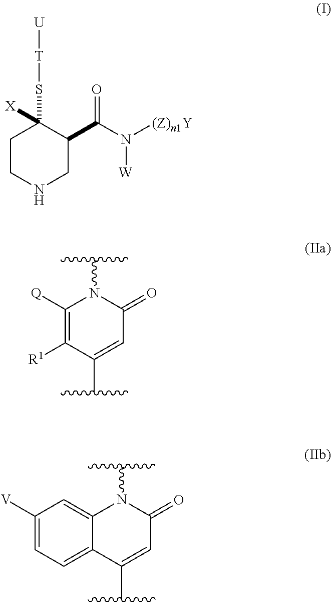

### CROSS-REFERENCE TO RELATED APPLICATIONS

This application claims the benefit of U.S. Provisional Application No. 61/201,367, filed Dec. 10, 2008.

### JOINT RESEARCH AGREEMENT

The claimed invention was made as a result of activities undertaken within the scope of a joint research agreement between Merck & Co., Inc. and Actelion Pharmaceuticals Ltd. The agreement was executed on Dec. 4, 2003. The field of the invention is described below.

### FIELD OF THE INVENTION

The invention relates to novel renin inhibitors of the general formula (I). The invention also concerns related aspects including processes for the preparation of the compounds, pharmaceutical compositions containing one or more compounds of formula (I) and especially their use as renin inhibitors in cardiovascular events and renal insufficiency.

### BACKGROUND OF THE INVENTION

In the renin-angiotensin system (RAS) the biologically active angiotensin II (Ang II) is generated by a two-step mechanism. The highly specific enzyme renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAS represents a major advance in the treatment of cardiovascular diseases. ACE inhibitors and AT₁ blockers have been accepted to treat hypertension (Waeber B. et al., “The renin-angiotensin system: role in experimental and human hypertension”, in Birkenhager W. H., Reid J. L. (eds): Hypertension, Amsterdam, Elsevier Science Publishing Co, 1986, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N. Engl. J. Med., 1992, 327, 669).

The rationale to develop renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be by-passed by chymase, a serine protease (Husain A., J. Hypertens., 1993, 11, 1155). In patients, inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the AT₁ receptor (e.g. by losartan) on the other hand overexposes other AT-receptor subtypes (e.g. AT₂) to Ang II, whose concentration is significantly increased by the blockade of AT₁ receptors. In summary, renin inhibitors are expected to demonstrate a different pharmaceutical profile than ACE inhibitors and AT₁ blockers with regard to efficacy in blocking the RAS and in safety aspects.

The present invention relates to the identification of renin inhibitors of a non-peptidic nature and of low molecular weight. Described are orally active renin inhibitors of long duration of action which are active in indications beyond blood pressure regulation where the tissular renin-chymase system may be activated leading to pathophysiologically altered local functions such as renal, cardiac and vascular remodeling, atherosclerosis, and possibly restenosis. The compounds described in this invention represent a novel structural class of renin inhibitors.

### SUMMARY OF THE INVENTION

The present invention is directed to certain compounds and their use in the inhibition of the renin enzyme, including treatment of conditions known to be associated with the renin system.

The invention in particular is directed to compounds of Formula I:

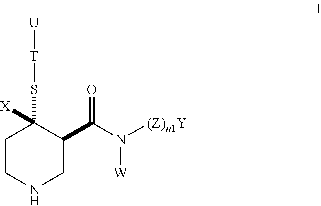

and optically pure enantiomers, mixtures of enantiomers such as racemates, diastereomers, mixtures of diastereomers, diastereomeric racemates, mixtures of diastereomeric racemates, meso-forms, salts, solvates, and morphological forms thereof, wherein constituent members are provided herein.

### DETAILED DESCRIPTION OF THE DISCLOSURE

The present invention provides compounds having Formula I:

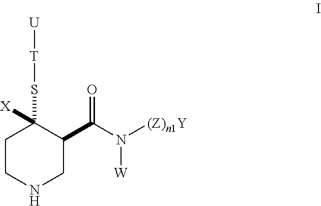

or a pharmaceutically acceptable salt thereof, or an optical isomer thereof, wherein:

S is

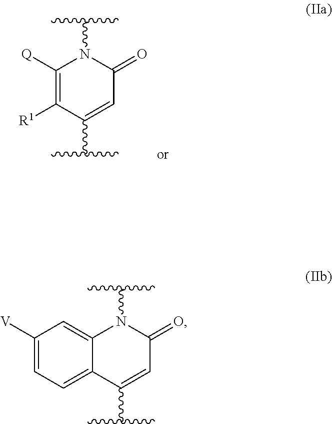

wherein the nitrogen atom in IIa and IIb above is attached to T;

T is a bond; —(CH₂)ᵣ—; —(CH₂)ᵣ-A-(CH₂)ₛ—; or —(CH₂)ᵣ-A-(CH₂)ᵣ—B—;

A and B are independently selected from the group consisting of —O—, —S—, —S(O)— and —S(O)₂—;

r is the integer 2, 3, 4, or 5;

s is the integer 0, 1, or 2;

U is unsubstituted aryl; mono-, di-, tri- or tetra-substituted aryl wherein the substituents are independently selected from the group consisting of halogen, alkyl, alkoxy, cyano and —CF₃; or mono-, di-, or tri-substituted heteroaryl wherein the substituents are independently selected from the group consisting of halogen, alkyl, alkoxy, cyano and —CF₃;

V is selected from the group consisting of: hydrogen, halogen, C₁-C₆ alkyl, C₃-C₆ cycloalkyl, C₂-C₆ alkenyl, C₃-C₆ cycloalkenyl, C₂-C₆ alkynyl, cyano and C₁-C₅ alkoxy,
wherein said alkyl, cycloalkyl, alkenyl, cycloalkenyl, alkynyl and alkoxy are optionally substituted with 1-3 substituents, each of which is independently selected from the group consisting of: halogen, C₁-C₅ alkyl, C₂-C₅ alkenyl, cyano and C₁-C₅ alkoxy, wherein each of the foregoing alkyl, alkenyl and alkoxy substituents is optionally substituted with 1-3 halogens;

Q and R¹ are independently selected from the group consisting of: hydrogen, halogen, C₁-C₅ alkyl, C₃-C₈ cycloalkyl, C₂-C₅ alkenyl, C₃-C₈ cycloalkenyl, C₂-C₅ alkynyl, cyano and C₁-C₅ alkoxy, aryl and heteroaryl;
wherein said aryl and heteroaryl are optionally substituted with 1-4 halogens,
wherein said alkyl, cycloalkyl, alkenyl, cycloalkenyl, alkynyl and alkoxy are optionally substituted with 1-3 substituents, each of which is independently selected from the group consisting of: halogen, C₁-C₅ alkyl, C₂-C₅ alkenyl, cyano and C₁-C₅ alkoxy, wherein each of the foregoing alkyl, alkenyl and alkoxy substituents is optionally substituted with 1-3 halogens;

W is cyclopropyl, unsubstituted or mono-, di-, tri-, tetra- or penta-substituted with halogen, specific embodiments of which the halogen is fluorine;

X is selected from the group consisting of: OR², R², —(C₁-C₅ alkylene)-(O)₀₋₁-aryl and —(C₁-C₅ alkylene)-(O)₀₋₁-heteroaryl,
wherein R² is selected from the group consisting of: hydrogen, C₁-C₅ alkyl, C₃-C₈ cycloalkyl, C₂-C₅ alkenyl, C₃-C₈ cycloalkenyl, C₂-C₅ alkynyl, C₁-C₅-cyano, —(C₁-C₅ alkylene)-O—R³, —(C₁-C₅ alkylene)-N(—R³)—C(═O)—(C₁-C₅ alkyl), —(C₁-C₅ alkylene)-C(═O)—N(—R³)—(C₁-C₅ alkyl), —(C₁-C₅ alkylene)-N(—R³)—C(═O)—O—(C₁-C₅ alkyl), —(C₁-C₅ alkylene)-O—C(═O)—N(—R³)—(C₁-C₅ alkyl); —(C₁-C₅ alkylene)-N(—R³)—(C₁-C₅ alkyl), —(C₁-C₅ alkylene)-S—(C₁-C₅ alkyl), —(C₁-C₅ alkylene)-S(═O)—(C₁-C₅ alkyl) and —(C₁-C₅ alkylene)-S(═O)₂—(C₁-C₅ alkyl),
wherein R², except hydrogen, is optionally substituted with 1-3 substituents, independently selected from the group consisting of: halogen, C(═O)OH, C₁-C₅ alkyl, C₂-C₅ alkenyl, and C₁-C₅ alkoxy, wherein each of the alkyl, alkenyl, and alkoxy substituents is optionally substituted with 1-3 halogens,
wherein the heteroaryl of the —(C₁-C₅ alkylene)-(O)₀₋₁-heteroaryl contains 1-3 heteroatoms, independently selected from the group consisting of: N, O and S, wherein each N is optionally in the form of an oxide and each S is optionally in the form of an oxide selected from the group consisting of: S(═O) and S(═O)₂,
wherein the aryl and heteroaryl of —(C₁-C₅ alkylene)-(O)₀₋₁-aryl and —(C₁-C₅ alkylene)-(O)₀₋₄-heteroaryl, respectively, are optionally substituted with 1-4 halogens, and
wherein R³ is selected from the group consisting of: hydrogen, C₁-C₆ alkyl, C₃-C₆ cycloalkyl, C₂-C₆ alkenyl, C₃-C₆ cycloalkenyl, and C₂-C₆ alkynyl, wherein each of the foregoing alkyl, cycloalkyl, alkenyl, cycloalkenyl and alkynyl substituents is optionally substituted with 1-3 halogens;

Z is C₁-C₂ alkylene optionally substituted with 1-2 substituents, independently selected from the group consisting of: halogen, C₁-C₃ alkyl and C₃ cycloalkyl, wherein the foregoing alkyl and cycloalkyl substituents are optionally substituted with 1-3 halogens;

n1 is 0 or 1;

Y is (i) a five- or six-membered saturated or unsaturated heterocyclic or carbocyclic monocyclic ring (“monocyclic ring”) or (ii) a fused ring system which is a five- or six-membered saturated or unsaturated heterocyclic or carbocyclic ring which is fused to a five- or six-membered saturated or unsaturated heterocyclic or carbocyclic ring (“fused ring”),
wherein the heterocyclic ring(s) of (i) or (ii) contain from 1-3 heteroatoms, independently selected from N, O and S, wherein each N is optionally in the form of an oxide and each S is optionally in the form of an oxide selected from the group consisting of: S(═O) and S(═O)₂,
wherein the heterocyclic or carbocyclic ring(s) of (i) or (ii) is optionally mono-, di-, tri-, tetra-, penta- or hexa-substituted, each substituent of which is independently selected from the group consisting of:
(1) halogen,
(2) —OH,
(3) —NH(R⁴),
(4) oxo,
(5) —C(═O)—R⁴,
(6) —O—C(═O)—R⁴,
(7) C₁-C₅ alkyl optionally substituted with 1-3 halogens,
(8) C₃-C₈ cycloalkyl optionally substituted with 1-3 halogens,
(9) C₂-C₅ alkenyl optionally substituted with 1-3 halogens,
(10) C₃-C₈ cycloalkenyl optionally substituted with 1-3 halogens,
(11) C₂-C₅ alkynyl optionally substituted with 1-3 halogens,
(12) C₁-C₅ alkoxy optionally substituted with 1-3 halogens,
(13) cyano,
(14) C₁-C₅-cyano optionally substituted with 1-3 halogens,
(15) —OCF₃,
(16) —C(R⁵)₃,
(17) —(C₁-C₅ alkylene)-OR⁶ optionally substituted with 1-3 halogens,
(18) —N(R⁴)—(C₁-C₅ alkylene)-OR⁶ optionally substituted with 1-3 halogens,
(19) —O—(C₁-C₅ alkylene)-OR⁶ optionally substituted with 1-3 halogens,
(20) —S—(C₁-C₅ alkylene)-OR⁶ optionally substituted with 1-3 halogens,
(21) —S(═O)—(C₁-C₅ alkylene)-OR⁶ optionally substituted with 1-3 halogens,
(22) —S(═O)₂—(C₁-C₅ alkylene)-OR⁶ optionally substituted with 1-3 halogens,
(23) —(C₁-C₅ alkylene)-N(R⁴)—C(═O)—(C₁-C₅ alkylene)-R⁶ optionally substituted with 1-3 halogens,
(24) —(C₁-C₅ alkylene)-N(R⁴)—C(═O)—OR⁶ optionally substituted with 1-3 halogens,
(25) —(C₁-C₅ alkylene)-N(R⁴)(R⁶) optionally substituted with 1-3 halogens,
(26) —O—(C₁-C₅ alkylene)-C(R⁴)₂—C(═O)OR⁶ optionally substituted with 1-3 halogens,
(27) —(C₁-C₅ alkylene)-C(R⁴)₂—C(═O)—OR⁶ optionally substituted with 1-3 halogens,
(28) —O—(C₁-C₅ alkylene)-morpholine optionally substituted with 1-3 halogens,
(29) —OC(═O)-morpholine,
(30) —SR⁶,
(31) —S(═O)—R⁶,
(32) —S(═O)₂—R⁶
(33) —N(R⁴)(R⁶),
(34) —(C₁-C₅ alkylene)-C(R⁴)₂—(R⁶) optionally substituted with 1-3 halogens,
(35) —(R⁷)₀₋₁R⁸,
(36) C₂-C₅ alkenyl-OR⁶ optionally substituted with 1-3 halogens,
(37) C₂-C₅ alkynyl-OR⁶ optionally substituted with 1-3 halogens,
(38) —(C₁-C₅ alkylene)-C(═O)—(C₁-C₅ alkylene)-R⁶ optionally substituted with 1-3 halogens,
(39) —(C₁-C₅ alkylene)-O—C(═O)—(C₁-C₅ alkylene)-R⁶ optionally substituted with 1-3 halogens,
(40) —(C₁-C₅ alkylene)-C(═O)—N(R⁴)(R⁶) optionally substituted with 1-3 halogens,
(41) —(C₁-C₅ alkylene)-O—C(═O)—N(R⁴)(R⁶) optionally substituted with 1-3 halogens,
(42) —(C₁-C₅ alkylene)-SR⁶ optionally substituted with 1-3 halogens,
(43) —(C₁-C₅ alkylene)-S(═O)—R⁶ optionally substituted with 1-3 halogens, and
(44) —(C₁-C₅ alkylene)-S(═O)₂—R⁶ optionally substituted with 1-3 halogens,
wherein R⁴ is selected from the group consisting of: hydrogen, C₁-C₆ alkyl, C₃-C₈ cycloalkyl, C₂-C₆ alkenyl, C₃-C₈ cycloalkenyl and C₂-C₆ alkynyl, wherein each of the foregoing alkyl, cycloalkyl, alkenyl, cycloalkenyl and alkynyl substituents is optionally substituted with 1-3 halogens,
wherein R⁵ is halogen,
wherein R⁶ is selected from the group consisting of: hydrogen, C₁-C₆ alkyl, C₃-C₈ cycloalkyl, C₂-C₆ alkenyl, C₃-C₈ cycloalkenyl and C₂-C₆ alkynyl, wherein each of the foregoing alkyl, cycloalkyl, alkenyl, cycloalkenyl and alkynl substituents is optionally substituted with 1-3 halogens,
wherein R⁷ is selected from the group consisting of: —C(H)(OH)—, —C(═O)—, —OC(═O)—, —C(═O)O—, —O—, —OC(═O)O—, C₁-C₅ alkylene, C₂-C₅ alkenylene, —N(R⁴)—, —S—, —S(═O)—, —S(═O)₂—, —N(R⁴)—C(═O)—, —C(═O)—N(R⁴)—, —OC(═O)—N(R⁴)—, —N(R⁴)—C(═O)O—, —N(R⁴)—S(═O)₂—, and —S(═O)₂—N(R⁴)—, wherein each of the foregoing alkylene and alkenylene substituents is optionally substituted with 1-3 halogens, and wherein R⁴ is defined above, and
wherein R⁸ is a five- or six-membered saturated or unsaturated heterocyclic or carbocyclic ring which is optionally mono-, di-, tri-, tetra- or penta-substituted, wherein each substituent is independently selected from the group consisting of: halogen, —OH, —SR⁴, —N(R⁴)(R⁶), C₁-C₅ alkyl, C₃-C₈ cycloalkyl, C₂-C₅ alkenyl, C₃-C₆ cycloalkenyl, C₂-C₅ alkynyl, C₁-C₅ alkoxy, cyano and C₁-C₅-cyano, wherein said heterocyclic ring contains from 1 to 3 heteroatoms, independently selected from N, O and S, wherein each N is optionally in the form of an oxide and each S is optionally is in the form of an oxide selected from the group consisting of: S(═O) and S(═O)₂, and wherein R⁴ and R⁶ are defined above.

In one embodiment, the invention provides compounds of Formula I, or a pharmaceutically acceptable salt thereof, or an optical isomer thereof, wherein the monocyclic or fused ring(s) of Y (i) or (ii), respectively, is selected from the following:

TABLE 3

| | |
|---|---|
| 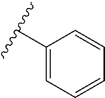 | 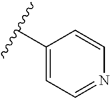 |
| 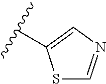 | 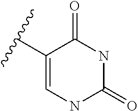 |
| 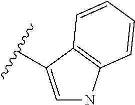 | 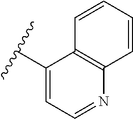 |
| 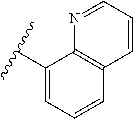 | 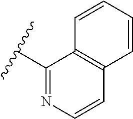 |
| 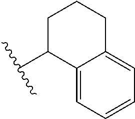 | 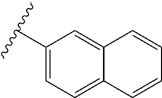 |
| 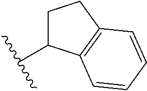 | 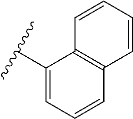 |
| 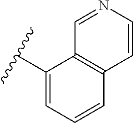 | 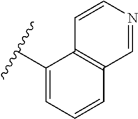 |
| 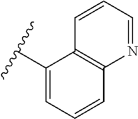 | |

optionally mono-, di-, tri-, tetra- or penta-substituted as described above for Formula I.

In another embodiment, T is —CH₂CH₂O—, or —CH₂CH₂CH₂O—, wherein the bivalent radical is linked to the group U of formula (I) via an oxygen atom.

In another embodiment of the invention, U is a mono-, di-, tri- or tetra-substituted aryl. In specific embodiments, U is a mono-, di-, or tri-substituted phenyl wherein the substituents are independently selected from the group consisting of halogen, C₁-C₆ alkyl, C₁-C₆ alkoxy, and —CF₃. In specific embodiments, the substituents are independently selected from the group consisting of halogen and C₁-C₆ alkyl. In specific embodiments, U represents 2,6-dichloro-4-methyl-phenyl, and all other variables are as previously defined.

In another embodiment, the invention provides compounds of Formula I, or a pharmaceutically acceptable salt thereof, or an optical isomer thereof, wherein Q and R¹ are independently selected from the group consisting of: H, —OCH₂OCH₃ and —CH₃.

In another embodiment, the invention provides compounds of Formula I, or a pharmaceutically acceptable salt thereof, or an optical isomer thereof, wherein V is hydrogen or halogen. In specific embodiments, the invention provides compounds of Formula I, or a pharmaceutically acceptable salt thereof, or an optical isomer thereof, wherein V is H or Cl.

In another embodiment, the invention provides compounds of Formula I, or a pharmaceutically acceptable salt thereof, or an optical isomer thereof, wherein W is cyclopropyl.

In another embodiment, the invention provides compounds of Formula I, or a pharmaceutically acceptable salt thereof, or an optical isomer thereof, wherein X is H, —OH or —OCH₃.

In another embodiment, the invention provides compounds of Formula I, or a pharmaceutically acceptable salt thereof, or an optical isomer thereof, wherein (Z)ₙ₁ is —CH₂— or a bond.

In another embodiment, the invention provides compounds of Formula I, or a pharmaceutically acceptable salt thereof, or an optical isomer thereof, wherein Y is

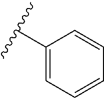

optionally mono-, di-, tri-, tetra- or penta-substituted as described in Formula I.

In another embodiment, the invention provides compounds of Formula I, or a pharmaceutically acceptable salt thereof, or an optical isomer thereof, wherein:

S is

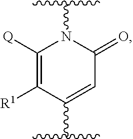

wherein the nitrogen atom in S above is attached to T,

U is a phenyl ring, optionally mono-, di-, tri-, tetra-, or penta-substituted wherein substituents are independently selected from the group consisting of: halogen, alkyl, alkoxy and CF₃,

W is cyclopropyl,

X is hydrogen, OH or methoxy,

Z is —CH₂—,

n1 is 1,

Y is

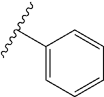

optionally mono-, di-, tri-, tetra- or penta-substituted as described in Formula I.,

wherein all other variables are as described above for Formula I.

In another embodiment, the invention provides compounds of Formula I, or a pharmaceutically acceptable salt thereof, or an optical isomer thereof, wherein:

S is

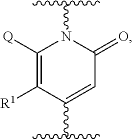

wherein the nitrogen atom in S above is attached to T,

T is —(CH₂)ᵣ—O—,

U is a phenyl ring, optionally mono-, di-, tri-, tetra-, or penta-substituted wherein substituents are independently selected from the group consisting of: halogen, alkyl, alkoxy and CF₃,

W is cyclopropyl,

X is hydrogen, OH or methoxy,

Z is —CH₂—,

n1 is 1,

Y is

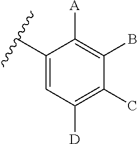

wherein:

A is selected from the group consisting of:
(1) hydrogen,
(2) halogen,
(3) C₁-C₅ alkyl,
(4) C₁-C₅ alkoxy, and
(5) —S—(CH₂)₀₋₃—CH₃,
wherein (3) and (4) are optionally substituted with 1-3 halogens,

B is selected from the group consisting of:
(1) hydrogen,
(2) halogen,
(3) C₁-C₅ alkyl,
(4) C₁-C₅ alkoxy,
(5) —OH,
(6) —CF₃,
(7) —C(═O)—CH₃,
(8) —O—(C₁-C₅ alkylene)-O-cyclopropyl,
(9) —O—(C₁-C₅ alkylene)-O—(CH₂)₀₋₂—CH₃,
(10) —(C₁-C₅ alkylene)-O—(CH₂)₀₋₂—CH₃,
(11) —OC(═O)-morpholine,
(12) —O—(C₁-C₅ alkylene)-morpholine,
(13) —O—(C₁-C₅ alkylene)-C(CH₃)₂—C(═O)OH,
(14) —O—(C₁-C₅ alkylene)-C(CH₃)₂—C(═O)OCH₃,

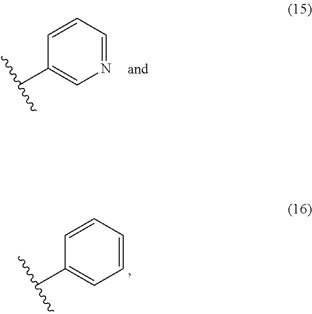

wherein (3), (4), (8), (9), (10), (12), (13), (14), (15) and (16) are optionally substituted with 1-3 halogens,

C is selected from the group consisting of:
(1) hydrogen,
(2) halogen,
(3) C₁-C₅ alkyl optionally substituted with 1-3 halogens, and
(4) C₁-C₅ alkoxy optionally substituted with 1-3 halogens, and

D is selected from the group consisting of:
(1) hydrogen,
(2) halogen,
(3) C₁-C₅ alkyl,
(4) C₁-C₅ alkoxy,
(5) C₁-C₅-cyano,
(6) C₂-C₅ alkenylene-O—(CH₂)₀₋₂—CH₃,
(7)-(C₁-C₅ alkylene)-N(H)—C(═O)—O—(CH₂)₀₋₂—CH₃,
(8) —(C₁-C₅ alkylene)-N(H)—C(═O)—(CH₂)₀₋₂—CH₃,
(9) —(C₁-C₅ alkylene)-O—CHF₂,
(10) —(C₁-C₅ alkylene)-O—(CH₂)₀₋₂—CH₃,
(11) —O—(C₁-C₅ alkylene)-O—(CH₂)₀₋₂—CH₃,
(12) —(C₁-C₅ alkylene)-OH,
(13) —S—(C₁-C₅ alkylene)-OH,
(14) —SCF₃
(15) —N(H)—(C₁-C₅ alkylene)-O—(CH₂)₀₋₂—CH₃, and
(16)

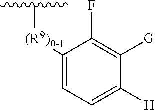

wherein F, G and H are independently selected from the group consisting of: hydrogen, halogen and C₁-C₃ alkyl optionally substituted with 1-3 halogens, and
wherein R⁹ is selected from the group consisting of: —CH₂—, —C(H)(OH)— and —C(═O)—,
wherein (3), (4), (5), (6), (7), (8), (9), (10), (11), (12), (13) and (15) are optionally substituted with 1-3 halogens, and

wherein all other variables are as described above for Formula I.

In another embodiment, the invention provides compounds of Formula I, or a pharmaceutically acceptable salt thereof, or an optical isomer thereof, wherein:

S is

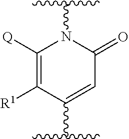

wherein the nitrogen atom in S above is attached to T,

T is —(CH₂)ᵣ—O—,

U is a phenyl ring, optionally mono-, di-, tri-, tetra-, or penta-substituted wherein substituents are independently selected from the group consisting of: halogen, alkyl, alkoxy and CF₃,

W is cyclopropyl,

X is hydrogen, OH or methoxy,

Z is —CH₂—,

n1 is 1,

Y is

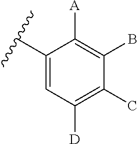

wherein:

A is selected from the group consisting of: hydrogen, halogen, C₁-C₅ alkyl and C₁-C₅ alkoxy,

B is selected from the group consisting of: hydrogen, halogen, C₁-C₅ alkyl, C₁-C₅ alkoxy and —O—(C₁-C₅ alkylene)-O—(CH₂)₀₋₂—CH₃,

C is selected from the group consisting of: hydrogen, halogen, C₁-C₅ alkyl and C₁-C₅ alkoxy,

D is selected from the group consisting of: hydrogen, halogen, C₁-C₅ alkyl, C₁-C₅ alkoxy and —(C₁-C₅ alkylene)-O—(CH₂)₀₋₂—CH₃,
wherein the alkyl or alkoxy groups of A-D are optionally substituted with 1-3 halogens, and

wherein all other variables are as described above for Formula I.

The compounds of Formula I above, and pharmaceutically acceptable salts thereof, are renin inhibitors. The compounds are useful for inhibiting renin and treating conditions such as hypertension.

Any reference to a compound of formula (I) is to be understood as referring also to optically pure enantiomers, mixtures of enantiomers such as racemates, diastereomers, mixtures of diastereomers, diastereomeric racemates, mixtures of diastereomeric racemates, meso-forms, as well as salts (especially pharmaceutically acceptable salts) and solvates (including hydrates) of such compounds, and morphological forms, as appropriate and expedient. The present invention encompasses all these forms. Mixtures are separated in a manner known per se, e.g. by column chromatography, thin layer chromatography (TLC), high performance liquid chromatography (HPLC), or crystallization. The compounds of the present invention may have chiral centers, e.g. one chiral center (providing for two stereoisomers, (R) and (S)), or two chiral centers (providing for up to four stereoisomers, (R,R), (S,S), (R,S), and (S,R)). This invention includes all of these optical isomers and mixtures thereof. Unless specifically mentioned otherwise, reference to one isomer applies to any of the possible isomers. Whenever the isomeric composition is unspecified, e.g., when bonds to a chiral carbon are depicted as straight lines, it is understood that both (R) and (S) configurations of that chiral carbon and, hence, both enantiomers and mixtures thereof are represented.

In addition, compounds with carbon-carbon double bonds may occur in Z- and E-forms with all isomeric forms of the compounds being included in the present invention.

Compounds of the invention also include nitrosated compounds of formula (I) that have been nitrosated through one or more sites such as oxygen (hydroxyl condensation), sulfur (sulfydryl condensation) and/or nitrogen. The nitrosated compounds of the present invention can be prepared using conventional methods known to one skilled in the art. For example, known methods for nitrosating compounds are described in U.S. Pat. Nos. 5,380,758, 5,703,073, 5,994,294, 6,242,432 and 6,218,417; WO 98/19672; and Oae et al., Org. Prep. Proc. Int., 15(3): 165-198 (1983).

Salts are preferably the pharmaceutically acceptable salts of the compounds of Formula (I). The expression “pharmaceutically acceptable salts” encompasses either salts with inorganic acids or organic acids like hydrochloric acid, hydrobromic acid, hydroiodic acid, sulfuric acid, sulfamic acid, phosphoric acid, nitric acid, phosphorous acid, nitrous acid, citric acid, formic acid, acetic acid, oxalic acid, maleic acid, lactic acid, tartaric acid, fumaric acid, benzoic acid, mandelic acid, cinnamic acid, palmoic acid, stearic acid, glutamic acid, aspartic acid, methanesulfonic acid, ethanesulfonic acid, ethanedisulfonic acid, p-toluenesulfonic acid, salicylic acid, succinic acid, trifluoroacetic acid, and the like that are non toxic to living organisms or, in case the compound of formula (I) is acidic in nature, with an inorganic base like an alkali or earth alkali base, e.g. sodium hydroxide, potassium hydroxide, calcium hydroxide and the like. For other examples of pharmaceutically acceptable salts, reference can be made notably to “Salt selection for basic drugs”, Int. J. Pharm. (1986), 33, 201-217.

The invention also includes derivatives of the compound of Formula I, acting as prodrugs. These prodrugs, following administration to the patient, are converted in the body by normal metabolic processes to the compound of Formula I. Such prodrugs include those that demonstrate enhanced bioavailability (see Table 4 below), tissue specificity, and/or cellular delivery, to improve drug absorption of the compound of Formula I. The effect of such prodrugs may result from modification of physicochemical properties such as lipophilicity, molecular weight, charge, and other physicochemical properties that determine the permeation properties of the drug.

The general terms used hereinbefore in Formula I and hereinafter preferably have, within this disclosure, the following meanings, unless otherwise indicated. Where the plural form is used for compounds, salts, pharmaceutical compositions, diseases and the like, this is intended to mean also a single compound, salt, or the like.

The term “alkyl”, alone or in combination with other groups, unless indicated otherwise, means saturated, straight and branched chain groups with one to six carbon atoms (which may be represented by “C₁₋₆ alkyl” or “C₁-C₆ alkyl”). When the intended meaning is other than this, for example, when the number of carbon atoms is in the range of one to four carbon atoms, this meaning is represented in like fashion as “C₁₋₄ alkyl” or “C₁-C₄ alkyl”. Examples of alkyl groups are methyl, ethyl, n-propyl, iso-propyl, n-butyl, iso-butyl, sec-butyl, tert-butyl, pentyl, hexyl and heptyl. The methyl, ethyl and isopropyl groups are preferred.

Structural depictions of compounds may show a terminal methyl group as “—CH₃”, “CH₃”, “-Me”, “Me” or

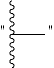

(i.e., these have equivalent meanings). A terminal ethyl group may be depicted as “—CH₂CH₃”, “CH₂CH₃”, “-Et”, “Et” or

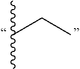

(i.e., these have equivalent meanings).

The term “alkylene” refers to any divalent linear or branched chain aliphatic hydrocarbon radical having a number of carbon atoms in the specified range. Thus, for example, “—C₁-C₆ alkylene-” refers to any of the C₁ to C₆ linear or branched alkylenes, and “—C₁-C₄ alkylene-” refers to any of the C₁ to C₄ linear or branched alkylenes. A class of alkylenes of particular interest with respect to the invention is —(CH₂)₁₋₆—, and sub-classes of particular interest include —(CH₂)₁₋₄—, —(CH₂)₁₋₃—, —(CH₂)₁₋₂—, and —CH₂—. Another sub-class of interest is an alkylene selected from the group consisting of —CH₂—, —CH(CH₃)—, and —C(CH₃)₂—. Expressions such as “C₁-C₄ alkylene-phenyl” and “C₁-C₄ alkyl substituted with phenyl” have the same meaning and are used interchangeably.

The term “alkenyl”, alone or in combination with other groups, unless indicated otherwise, means unsaturated (i.e., having at least one double bond) straight and branched chain groups with two to six carbon atoms (which may be represented by “C₂₋₆ alkenyl” or “C₂-C₆ alkenyl”). When the intended meaning is other than this, for example, when the number of carbon atoms is in the range of two to four carbon atoms, this meaning is represented in like fashion as “C₂₋₄ alkenyl” or “C₂-C₄ alkenyl”.

The term “alkenylene” refers to any divalent linear or branched chain aliphatic mono-unsaturated hydrocarbon radical having a number of carbon atoms in the specified range.

The term “alkynyl”, alone or in combination with other groups, unless indicated otherwise, means unsaturated (i.e., having at least one triple bond) straight and branched chain groups with two to six carbon atoms (which may be represented by “C₂₋₆ alkynyl” or “C₂-C₆ alkynyl”). When the intended meaning is other than this, for example, when the number of carbon atoms is in the range of two to four carbon atoms, this meaning is represented in like fashion as “C₂₋₄ alkynyl” or “C₂-C₄ alkynyl”.

The term “alkoxy”, alone or in combination with other groups, refers to an R—O— group, wherein R is an alkyl group. Examples of alkoxy groups are methoxy, ethoxy, propoxy, iso-propoxy, iso-butoxy, sec-butoxy and tert-butoxy.

The term “hydroxy-alkyl”, alone or in combination with other groups, refers to an HO—R— group, wherein R is an alkyl group. Examples of hydroxy-alkyl groups are HO—CH₂—, HO—CH₂CH₂—, HO—CH₂CH₂CH₂— and CH₃CH(OH)—.

The term “halogen” means fluorine, chlorine, bromine or iodine, preferably fluorine, chlorine or bromine, especially fluorine or chlorine.

The term “cycloalkyl”, alone or in combination with other groups, unless indicated otherwise, means a saturated cyclic hydrocarbon ring system with 3 to 8 carbon atoms, e.g., cyclopropyl, cyclobutyl, cyclopentyl, cyclohexyl, cycloheptyl and cyclooctyl. This may be represented by “C₃₋₈ cycloalkyl” or “C₃-C₈ cycloalkyl”). When the intended meaning is other than this, for example, when the number of carbon atoms is in the range of three to six carbon atoms, this meaning is represented in like fashion as “C₃₋₆ cycloalkyl” or “C₃-C₆ cycloalkyl”.

The term “carbocycle” (and variations thereof such as “carbocyclic” or “carbocyclyl”) as used herein, unless otherwise indicated, refers to a C₃ to C₈ monocyclic saturated or unsaturated ring. The carbocycle may be attached to the rest of the molecule at any carbon atom which results in a stable compound. Saturated carbocyclic rings are also referred to as cycloalkyl rings, e.g., cyclopropyl, cyclobutyl, etc.

The term “heterocycle” (and variations thereof such as “heterocyclic” or “heterocyclyl”) broadly refers to a stable 4- to 8-membered, saturated or unsaturated monocyclic ring which contains one or more heteroatoms (e.g., from 1 to 6 heteroatoms, or from 1 to 4 heteroatoms) selected from N, O and S and a balance of carbon atoms (typically at least one carbon atom); wherein any one or more of the nitrogen and sulfur heteroatoms is optionally oxidized, and any one or more of the nitrogen heteroatoms is optionally quaternized. Unless otherwise specified, the heterocyclic ring may be attached at any heteroatom or carbon atom, provided that attachment results in the creation of a stable structure. Unless otherwise specified, when the heterocyclic ring has substituents, it is understood that the substituents may be attached to any atom in the ring, whether a heteroatom or a carbon atom, provided that a stable chemical structure results.

The term “aryl”, alone or in combination, relates to a phenyl, naphthyl or indanyl group, preferably a phenyl group. The abbreviation “Ph” represents phenyl.

The term “heteroaryl”, alone or in combination, means six-membered aromatic rings containing one to four nitrogen atoms; benzofused six-membered aromatic rings containing one to three nitrogen atoms; five-membered aromatic rings containing one oxygen, one nitrogen or one sulfur atom; benzofused five-membered aromatic rings containing one oxygen, one nitrogen or one sulfur atom; five-membered aromatic rings containing two heteroatoms independently selected from oxygen, nitrogen and sulfur and benzofused derivatives of such rings; five-membered aromatic rings containing three nitrogen atoms and benzofused derivatives thereof; a tetrazolyl ring; a thiazinyl ring; or coumarinyl. Examples of such ring systems are furanyl, thienyl, pyrrolyl, pyridinyl, pyrimidinyl, indolyl, quinolinyl, isoquinolinyl, imidazolyl, triazinyl, thiazolyl, isothiazolyl, pyridazinyl, pyrazolyl, oxazolyl, isoxazolyl, benzothienyl, quinazolinyl and quinoxalinyl.

Specific examples of compounds of formula I, and pharmaceutically acceptable salts thereof, include those listed below:
trans-N-Cyclopropyl-4-{1-[2-(2,6-dichloro-4-methylphenoxy)ethyl]-2-oxo-1,2-dihydro-4-pyridinyl}-4-hydroxy-N-[3-(2-methoxyethoxy)-5-(3-methoxypropyl)benzyl]-3-piperidinecarboxamide
trans-N-Cyclopropyl-4-{1-[2-(2,6-dichloro-4-methylphenoxy)ethyl]-2-oxo-1,2-dihydro-4-pyridinyl}-4-methoxy-N-[3-(2-methoxyethoxy)-5-(3-methoxypropyl)benzyl]-3-piperidinecarboxamide
trans-N-Cyclopropyl-4-{1-[3-(2,6-dichloro-4-methylphenoxy)propyl]-2-oxo-1,2-dihydro-4-pyridinyl}-4-hydroxy-N-[3-(2-methoxyethoxy)-5-(3-methoxypropyl)benzyl]-3-piperidinecarboxamide
trans-N-Cyclopropyl-4-{1-[3-(2,6-dichloro-4-methylphenoxy)propyl]-2-oxo-1,2-dihydro-4-pyridinyl}-4-methoxy-N-[3-(2-methoxyethoxy)-5-(3-methoxypropyl)benzyl]-3-piperidinecarboxamide
trans-N-[2-Chloro-5-(2-methoxyethyl)benzyl]-N-cyclopropyl-4-{1-[3-(2,6-dichloro-4-methylphenoxy)propyl]-2-oxo-1,2-dihydro-4-pyridinyl)-4-hydroxy-3-piperidinecarboxamide

The present invention also encompasses a pharmaceutical formulation comprising a pharmaceutically acceptable carrier and the compound of Formula I or a pharmaceutically acceptable crystal form or hydrate thereof. A preferred embodiment is a pharmaceutical composition of the compound of Formula I, comprising, in addition, a second agent.

### LIST OF ABBREVIATIONS

BINAP 2,2′-bis(diphenylphosphino)-1,1′-binaphthyl
BOC t-butyloxycarbonyl
BSA bovine serum albumin
COD 1,5-cyclooctadiene
DBU 1,8-diazabicyclo[5.4.0]undec-7-ene
DCM dichloromethane
DIBAl-H diisobutylaluminum hydride
DMAP 4-dimethylamino pyridine
DME 1,2-dimethoxyethane

### DMF N,N-dimethylformamide

DMP Dess-Martin periodinane
DMSO dimethylsulfoxide
DPPB 1,4-bis(diphenylphosphino)butane
DPPF 1,1′-bis(diphenylphosphino)ferrocene
EDTA ethylenediaminetetraacetic acid
EIA enzyme immunoassay
Et₂O diethylether
EtOAc ethyl acetate
HATU O-(7-azabenzotriazol-1-yl)-N,N,N′,N′-tetramethyluronium hexafluorophosphate
Hex hexanes
KHMDS potassium hexamethyldisilazide
mCPBA meta-chloroperbenzoic acid
MeOH methanol
NBS N-bromo succinimide

### NMO N-methylmorpholine-N-oxide

n-PrOH n-propanol
PBS phosphate-buffered saline
PG protecting group
PPh3 triphenylphosphine
RT room temperature
TBAF tetrabutylammonium fluoride
TFA trifluoroacetic acid
THF tetrahydrofuran
TMEDA N,N,N′,N′-tetramethylethylenediamine
Tol toluene

Unless expressly stated to the contrary, all ranges cited herein are inclusive. For example, an alkyl group described as C₁-C₆ alkyl means the alkyl group can contain 1, 2, 3, 4, 5 or 6 carbon atoms.

When a given range includes 0 (e.g., (CH₂)₀₋₃), 0 implies a direct covalent bond.

When any variable occurs more than one time in any constituent or in any formula depicting and describing compounds of the invention, its definition on each occurrence is independent of its definition at every other occurrence.

Also, combinations of substituents and/or variables are permissible only if such combinations result in stable compounds.

The term “substituted” (e.g., as in “aryl which is optionally substituted with one or more substituents . . . ”) includes mono- and poly-substitution by a named substituent to the extent such single and multiple substitution (including multiple substitution at the same site) is chemically allowed and results in a stable compound.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

In compounds of the invention having pyridyl N-oxide moieties, the pyridyl-N-oxide portion is structurally depicted using conventional representations such as

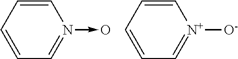

which have equivalent meanings.

The invention relates to a method for the treatment and/or prophylaxis of diseases which are related to hypertension, congestive heart failure, pulmonary hypertension, systolic hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, which method comprises administrating a compound as defined above to a human being or animal.

In another embodiment, the invention relates to a method for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, complications resulting from diabetes such as nephropathy, vasculopathy and neuropathy.

In another embodiment, the invention relates to a method for the treatment and/or prophylaxis of diseases, which are associated with a dysregulation of the renin-angiotensin system as well as for the treatment of the above-mentioned diseases.

The invention also relates to the use of compounds of formula (I) for the preparation of a medicament for the treatment and/or prophylaxis of the above-mentioned diseases.

Compounds of formula (I) or the above-mentioned pharmaceutical compositions are also of use in combination with other pharmacologically active compounds comprising ACE-inhibitors, neutral endopeptidase inhibitors, angiotensin II receptor antagonists, endothelin receptors antagonists, vasodilators, calcium antagonists, potassium activators, diuretics, sympatholitics, beta-adrenergic antagonists, alpha-adrenergic antagonists or with other drugs beneficial for the prevention or the treatment of the above-mentioned diseases.

The term “administration” and variants thereof (e.g., “administering” a compound) in reference to a compound of Formula I mean providing the compound or a prodrug of the compound to the individual in need of treatment or prophylaxis. When a compound of the invention or a prodrug thereof is provided in combination with one or more other active agents (e.g., an agent such as anangiotensin II receptor antagonist, ACE inhibitor, or other active agent which is known to reduce blood pressure), “administration” and its variants are each understood to include provision of the compound or prodrug and other agents at the same time or at different times. When the agents of a combination are administered at the same time, they can be administered together in a single composition or they can be administered separately.

As used herein, the term “composition” is intended to encompass a product comprising the specified ingredients in the specified amounts, as well as any product which results, directly or indirectly, from combining the specified ingredients in the specified amounts.

By “pharmaceutically acceptable” is meant that the ingredients of the pharmaceutical composition must be compatible with each other and not deleterious to the recipient thereof.

The term “subject” as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit renin and thereby elicit the response being sought (i.e., an “inhibition effective amount”). When the active compound (i.e., active ingredient) is administered as the salt, references to the amount of active ingredient are to the free form (i.e., the non-salt form) of the compound.

In a preferred embodiment, this amount is comprised between 1 mg and 1000 mg per day. In a particularly preferred embodiment, this amount is comprised between 1 mg and 500 mg per day. In a more particularly preferred embodiment, this amount is comprised between 1 mg and 200 mg per day.

In the method of the present invention (i.e., inhibiting renin), the compounds of Formula I, optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, mucosally (including sublingual, buccal, rectal, nasal or vaginal administrations), parenterally (including subcutaneous injection, bolus injection, intraarterial, intravenous, intramuscular, intrasternal injection or infusion administrations techniques), by inhalation spray, transdermal, such as passive or iontophoretic delivery, or topical administration, in the form of a unit dosage of a pharmaceutical composition containing an effective amount of the compound and conventional non-toxic pharmaceutically-acceptable carriers, adjuvants and vehicles. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990.

### Methods of Synthesis

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. LaRock, Comprehensive Organic Transformations, 2.sup.nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes and examples are merely illustrative of some methods by which the compounds of the present invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specifically stated otherwise, the experimental procedures were performed under the following conditions. Evaporation of solvent was carried out using a rotary evaporator under reduced pressure (600-4000 pascals: 4.5-30 mm Hg) with a bath temperature of up to 60° C. Reactions are typically run under nitrogen atmosphere at ambient temperature if not otherwise mentioned. Anhydrous solvent such as THF, DMF, Et₂O, DME and Toluene are commercial grade. Reagents are commercial grade and were used without further purification. Flash chromatography is run on silica gel (230-400 mesh). The course of the reaction was followed by either thin layer chromatography (TLC) or nuclear magnetic resonance (NMR) spectrometry and reaction times given are for illustration only. The structure and purity of all final products were ascertained by TLC, mass spectrometry, ¹H NMR and high-pressure liquid chromatography (HPLC). Chemical symbols have their usual meanings. The following abbreviations have also been used: v (volume), w (weight), b.p. (boiling point), m.p. (melting point), L (liter(s)), mL (milliliter(s)), g (gram(s)), mg (milligram(s)), mol (mole(s)), mmol (millimole(s)), eq. (equivalent(s)). Unless otherwise specified, all variables mentioned below have the meanings as provided above.

Generally, compounds of the present invention can be prepared via the coupling of an appropriately substituted pyridone I with an appropriately functionalized amine II, followed by the removal of the BOC-protecting group from amide III (Scheme 1).

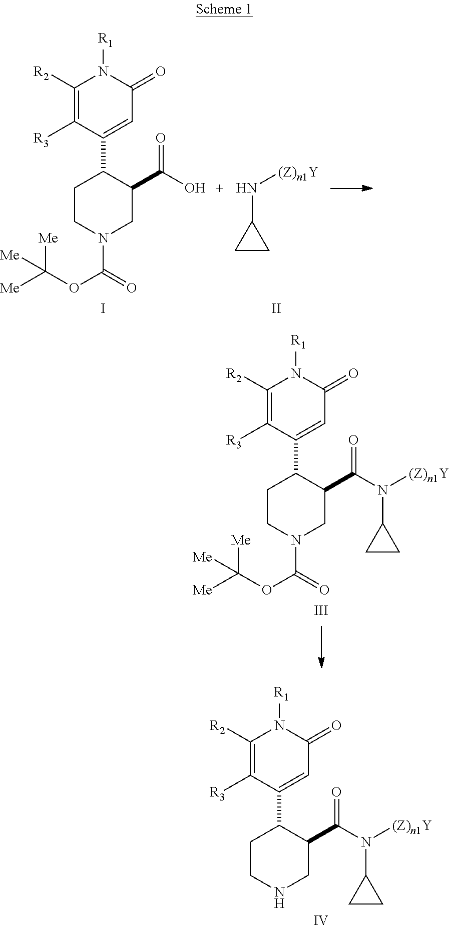

Synthesis of the requisite pyridone I can, for example, be performed as exemplified in Scheme 2. Typically, metal-catalyzed Suzuki coupling of boronate VI, prepared from known triflate V, with halide VII, can provide α,β-unsaturated ester VIII. Reduction of the double bond; most conveniently accomplished using either magnesium or samarium iodide, and subsequent base-mediated equilibration, would then afford saturated ester IX as a single diastereomer. Its conversion to the corresponding pyridone X can be realized in two steps via the initial treatment of ester IX with mCPBA; or an equivalent oxidant, followed by the reaction of the resulting pyridine N-oxide with TFAA in triethylamine; or an equivalent rearrangement promoter. Alternatively, for cases where the V group of ester IX is OBn, a simple hydrogenation under typical conditions would directly furnish pyridone X. Indeed, one can also access XI via N-alkylation of pyridone X with an appropriate reagent. Finally, saponification of pyridone XI would furnish pyridone I.

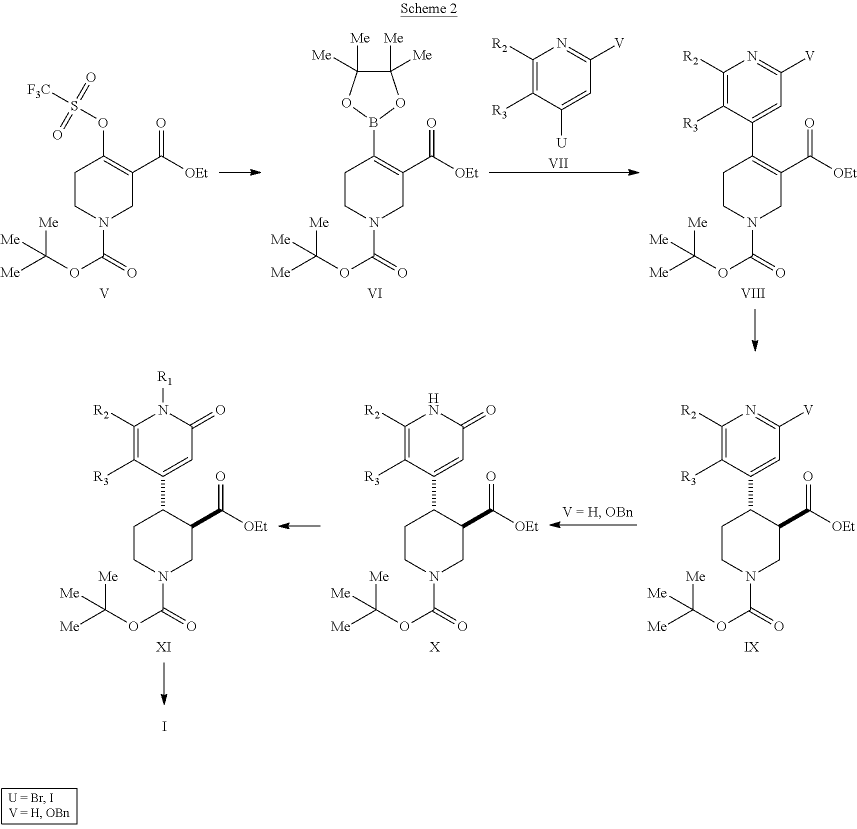

For halides VII used in the preparation of I where V is OBn, their synthesis can most readily be accomplished from the corresponding pyridone XIV using, for example, benzyl halide in the presence of silver carbonate (Scheme 3). For cases where pyridone XIV was neither commercially available nor known in the literature, the requisite compound could be prepared from its corresponding pyridine XII via the intermediacy of pyridine N-oxide XIII.

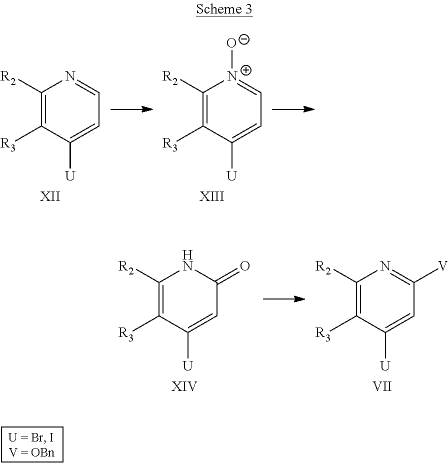

For compounds of the present invention bearing an alkoxy group at position 4 of the piperidine ring, they are most conveniently accessed via an initial amide formation between amine II and β-ketoester XV, followed by the addition of Gignard reagent derived from a suitably-protected and appropriately substituted hydroxypyridine. Functionalization of the resulting alcohol XVII, if necessary, would precede the conversion of the protected hydroxypyridine XXVIII into the desired pyridone XIX using chemistry described earlier. Finally, BOC removal can be accomplished under typical conditions (Scheme 4).

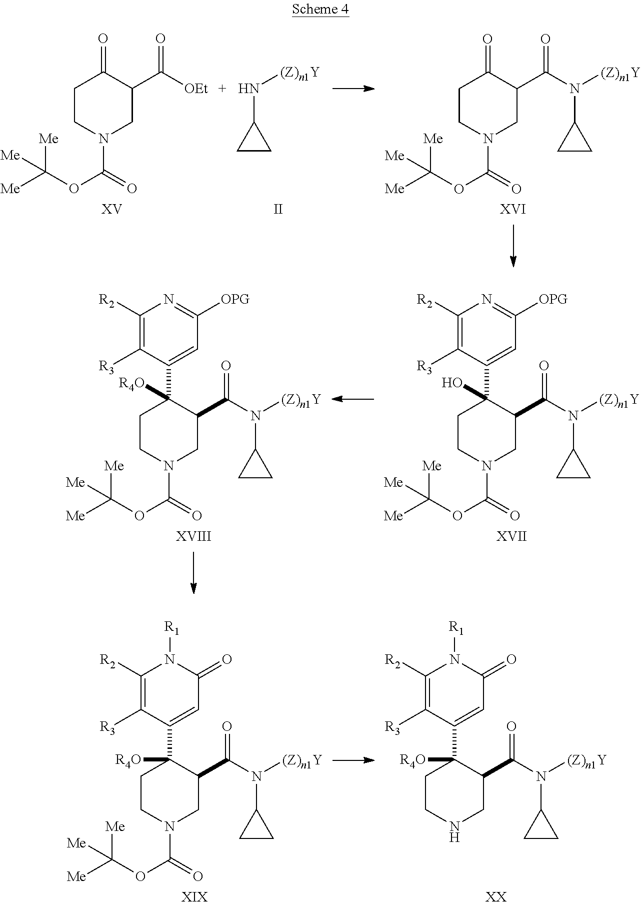

Representative cyclopropylamine building blocks are shown in Table 1.

TABLE 1

| Compound | Structure |
|---|---|
| Amine 1 | 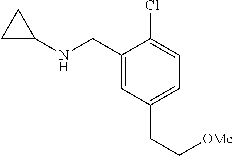 |
| Amine 2 | 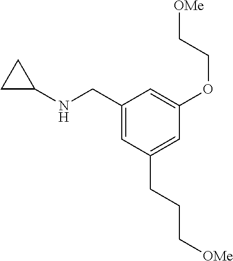 |

### Amine 1

### N-({2-Chloro-5-[2-(methyloxy)ethyl]phenyl}methyl)cyclopropanamine

Amine 1 was prepared according to the procedure described in WO 2007/009250 A1 patent.

### Amine 2

### N-({3-{[2-(Methyloxy)ethyl]oxy}-5-[3-(methyloxy)propyl]phenyl}methyl)cyclopropanamine

### Step 1: 3-Bromo-5-hydroxybenzaldehyde

To a toluene solution (1.6 M) of n-butyl lithium (2.5 M in hexane, 2.1 eq.) was added at −10° C. n-butyl magnesium chloride (2.0 M in THF, 0.6 eq.). The reaction mixture was stirred at −10° C. for 30 min before a toluene solution (0.7 M) of 3,5-dibromophenol (1 eq.) was added dropwise at −10° C. over a period of 35 min. After stirring at −10° C. for a further 30 min, the reaction mixture was cooled to −40° C. before DMF (20 eq.) was added dropwise over 20 min. The reaction mixture was then slowly warmed to RT and allowed to stir at RT for 1 h. The reaction was carefully quenched at 0° C. with 1 N aq. HCl and extracted with ether. The combined organic extracts were washed with water and brine, dried over MgSO₄ and filtered. Concentration of the filtrate in vacuo afforded a yellow solid. Recystallization of the crude product thus obtained from ether-hexane afforded the title compound as a beige powder.

### Step 2: 3-Hydroxy-5-[(1E)-3-(methyloxy)-1-propen-1-yl]benzaldehyde

3-Bomo-5-hydroxybenzaldehyde (1 eq.) from the previous step and 2-[(1E)-3-methoxyprop-1-en-1-yl]-4,4,5,5-tetramethyl-1,3,2-dioxaborolane (1 eq.) were combined in DMF (0.05 M). To this solution was then added palladium acetate (0.1 eq.), triphenylphosphine (0.2 eq.) and 2 M aq. sodium carbonate (4 eq.). The resulting suspension was heated at 80° C. for 16 h. The reaction mixture was quenched with 1 N aq. HCl and extracted with ether. The combined organic extracts were washed with water, sat. aq. NaHCO₃ and brine. Drying over MgSO₄, filtration and concentration of the filtrate in vacuo afforded the crude product as a brown tar. Further purification by way of flash chromatography (SiO₂, 4:1 (v/v) Hex:EtOAc→2:1 (v/v) Hex:EtOAc) afforded the title compound as a yellow oil.

### Step 3: 3-{[2-(Methyloxy)ethyl]oxy}-5-[(1E)-3-(methyloxy)-1-prop-1-en-1-yl]benzaldehyde

3-Hydroxy-5-[(1E)-3-(methyloxy)-1-propen-1-yl]benzaldehyde (1 eq.) from the previous step and 2-bromoethyl methyl ether (2.2 eq.) were combined in DMF. To this solution was then added cesium carbonate (2 eq.) and the reaction suspension was heated at 100° C. for 16 h. The resulting reaction mixture was quenched with water and back-extracted with ether. The combined organic extracts were washed further with water and brine, dried over MgSO₄, filtered and the filtrate concentrated in vacuo. Further purification of the crude product thus obtained by way of flash chromatography (SiO₂, 19:1 (v/v) Hex:EtOAc→1:1 (v/v) Hex:EtOAc) afforded the title compound as a yellow oil.

### Step 4: N-({3-{[2-(Methyloxy)ethyl]oxy}-5-[(1E)-3-(methyloxy)-1-propen-1-yl]phenyl}methyl)cyclopropanamine

To a solution of 3-{[2-(methyloxy)ethyl]oxy}-5-[(1E)-3-(methyloxy)-1-prop-1-en-1-yl]benzaldehyde (1 eq.) from the previous step (1 eq.) in dichloromethane (0.5 M) was added cyclopropylamine (2 eq.) and magnesium sulfate (1.5 eq.). The resulting suspension was stirred at RT for 12 h. The insolubles were removed via filtration. Concentration of the filtrate in vacuo afforded the crude imine as a yellow oil. This was then taken up in methanol (0.3 M) and then added at 0° C. sodium borohydride (1.5 eq.) portionwise over 5 min. The reaction mixture was slowly warmed to RT over 1 h and then stirred at RT for 2 h. After carefully quenching with sat. aq. NaHCO₃, the resulting mixture was extracted with ether. The combined organic extracts were washed with water and brine, dried over MgSO₄ and filtered. Concentration of the filtrate in vacuo afforded the title compound as a golden, yellow oil.

### Step 5: Amine 2

To a solution of N-({3-{[2-(methyloxy)ethyl]oxy}-5-[(1E)-3-(methyloxy)-1-propen-1-yl]phenyl}methyl)cyclopropanamine (1 eq.) from the previous step in EtOAc (0.04 M) was added palladium (10% w/w over activated carbon, 0.1 eq). The vessel was evacuated and back filled with hydrogen. The reaction suspension was then stirred under a balloon atmosphere of hydrogen for 1.5 h. The reaction was quenched with dichloromethane and filtered through a bed of celite. The insolubles were washed further with EtOAc and methanol. Concentration of the filtrate in vacuo afforded the title compound as a colorless oil.

The arene building blocks in Table 2 were synthesized as follows.

TABLE 2

| Compound | Structure |
|---|---|
| Arene 1 | 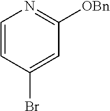 |

### Arene 1

### 2-(Benzyloxy)-4-bromopyridine

To a solution of 4-bromo-2-fluoropyridine (1 eq.), benzyl alcohol (1.2 eq.) and dibenzo-18-crown-6 (0.05 eq.) in toluene (0.4 M) was added potassium hydroxide (2 eq.). A Dean-Stark apparatus was then attached and the reaction suspension was heated at reflux for 3 h. After cooling to RT, the reaction mixture was diluted with hexanes and then filtered through a pad of celite. Concentration of the filtrate in vacuo afforded a yellow oil. Purification of the crude product thus obtained by way of column chromatography (SiO₂, 97:3 (v/v) Hex:Et₂O) afforded the title compound as a colorless oil.

Representative alkylation building blocks are shown in Table 3.

TABLE 3

| Compound | Structure |
|---|---|
| Mesylate 1 | 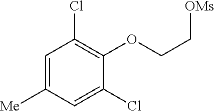 |
| Mesylate 2 | 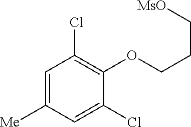 |

### Mesylate 1

### 2-(2,6-Dichloro-4-methylphenoxy)ethyl methanesulfonate

### Step 1: 2-(2,6-Dichloro-4-methylphenoxy)ethanol

2,6-Dichloro-4-methylphenol (1 eq.), ethylene carbonate (1 eq.) and imidazole (0.5% loading) were combined and heated at 150° C. for 4 h to afford the title compound as a brown oil.

### Step 2: Mesylate 1

To a dichloromethane solution (0.18 M) solution of 2-(2,6-dichloro-4-methylphenoxy)ethanol (1 eq.) from the previous step was added at −40° C. methanesulfonyl chloride (1.5 eq.) and triethylamine (3 eq.). The resulting mixture was then slowly warmed to 5° C. over 1.5 h. The reaction mixture was quenched with sat. aq. NH₄Cl and extracted with dichloromethane. The combined organic extracts were washed with water and brine. Drying over MgSO₄, filtration and concentration of the filtrate in vacuo afforded the crude product that could be further purified by way of flash chromatography (SiO₂, 4:1 (v/v) Hex: EtOAc→2:1 (v/v) Hex: EtOAc). The title compound was obtained as a pale yellow oil.

### Mesylate 2

### 3-(2,6-Dichloro-4-methylphenoxy)propyl methanesulfonate

### Step 1: tert-Butyl[3-(2,6-dichloro-4-methylphenoxy)propoxy]dimethylsilane

To a THF solution (0.23 M) of 2,6-dichloro-4-methylphenol (1 eq.), 3-{[tert-butyl(dimethyl)silyl]oxy}-1-propanol (1 eq.) and di-tert-butyl azodicarboxylate (1.3 eq.) was added tri-n-butylphosphine (1.5 eq.). The resulting suspension was stirred at RT for 16 h. The reaction mixture was the diluted with ether and washed sequentially with 10% aq. HCl, 1 N aq. NaOH, water and brine. Drying over MgSO₄, filtration and concentration of the filtrate in vacuo afforded the crude product that could be further purified by way of flash chromatography (SiO₂, 99:1 (v/v) Hex: Et₂O→97:3 (v/v) Hex: Et₂O). The title compound was obtained as a colourless oil.

### Step 2: 3-(2,6-Dichloro-4-methylphenoxy)-1-propanol

To a THF solution (0.25 M) solution of tert-butyl[3-(2,6-dichloro-4-methylphenoxy)propoxy]dimethylsilane (1 eq.) from the previous step was added tetrabutylammonium fluoride (1.0 M THF solution, 1.1 eq.). The resulting mixture was stirred at RT for 4 h. The reaction mixture was then quenched with sat. aq. NH₄Cl and extracted with ether. The combined organic extracts were washed with water and brine. Drying over MgSO₄, filtration and concentration of the filtrate in vacuo afforded the crude product that could be further purified by way of flash chromatography (SiO₂, 4:1 (v/v) Hex:EtOAc→2:1 (v/v) Hex: EtOAc) afforded the title compound as a colourless oil.

### Step 3: Mesylate 2

To a dichloromethane solution (0.15 M) solution of 3-(2,6-dichloro-4-methylphenoxy)-1-propanol (1 eq.) from the previous step was added at −40° C. methanesulfonyl chloride (1.5 eq.) and triethylamine (3 eq.). The resulting mixture was then slowly warmed to 5° C. over 1 h. The reaction mixture was quenched with sat. aq. NH₄Cl and extracted with dichloromethane. The combined organic extracts were washed with water and brine. Drying over MgSO₄, filtration and concentration of the filtrate in vacuo afforded the crude product that could be further purified by way of flash chromatography (SiO₂, 4:1 (v/v) Hex: EtOAc→2:1 (v/v) Hex: EtOAc). The title compound was obtained as a pale yellow oil.

### Example 1

### trans-N-Cyclopropyl-4-{1-[2-(2,6-dichloro-4-methylphenoxy)ethyl]-2-oxo-1,2-dihydro-4-pyridinyl}-4-hydroxy-N-[3-(2-methoxyethoxy)-5-(3-methoxypropyl)benzyl]-3-piperidinecarboxamide

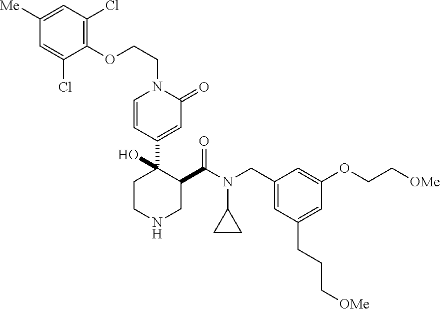

### Step 1: tert-Butyl 3-({cyclopropyl[3-(2-methoxyethoxy)-5-(3-methoxypropyl)benzyl]amino}carbonyl)-4-oxo-1-piperidinecarboxylate

1-tert-Butyl 3-ethyl 4-oxo-1,3-piperidinedicaroboxylate (1 eq.), Amine 2 (1 eq.) and DMAP (0.2 eq.) were heated at 140° C. for 5 h. Purification of the crude product thus obtained by way of column chromatography (SiO₂, 95:5→3:7 (v/v) Hex: EtOAc) followed by swishing in 9:1 (v/v) Hex: Et₂O afforded the title compound as a white solid.

### Step 2: tert-Butyl trans-4-[2-(benzyloxy)-4-pyridinyl]-3-({cyclopropyl[3-(2-methoxyethoxy)-5-(3-methoxypropyl)benzyl]amino}carbonyl)-4-hydroxy-1-piperidinecarboxylate

To a THF solution (0.08 M) of Arene 1 was added at −78° C. n-butyl lithium (2.5 M solution in hexanes, 2.1 eq.). After stirring at −78° C. for 30 min, solid magnesium bromide (2.5 eq.) was added in one rapid portion and the resulting mixture was stirred at −78° C. for 20 min. The reaction mixture was then slowly warmed to 0° C. over 30 min and tert-butyl 3-({cyclopropyl[3-(2-methoxyethoxy)-5-(3-methoxypropyl)benzyl]amino}carbonyl)-4-oxo-1-piperidinecarboxylate (1 eq.) from the previous step was added as a THF solution. The reaction mixture was then stirred at 0° C. for 1 h and at RT for 30 min. The reaction was then quenched with the addition of sat. aq. NH₄Cl and ether. The aqueous layer was separated and back-extracted with ether. The combined organic extracts were washed further with brine, dried over MgSO₄, filtered and the filtrate concentrated in vacuo. Purification of the crude product thus obtained by way of column chromatography (SiO₂, 96:4→93:7 (v/v) acetone: toluene) afforded the title compound.

### Step 3: tert-Butyl trans-3-({cyclopropyl[3-(2-methoxyethoxy)-5-(3-methoxypropyl)benzyl]amino}carbonyl)-4-hydroxy-4-(2-oxo-1,2-dihydro-4-pyridinyl)-1-piperidinecarboxylate

To a solution of tert-butyl trans-4-[2-(benzyloxy)-4-pyridinyl]-3-({cyclopropyl[3-(2-methoxyethoxy)-5-(3-methoxypropyl)benzyl]amino}carbonyl)-4-hydroxy-1-piperidinecarboxylate

(1 eq.) from the previous step in EtOAc (0.1 M) was added palladium (10% w/w on carbon, 0.5 eq.) and acetic acid (1.1 eq.). The resulting suspension was stirred under a balloon atmosphere of hydrogen for 4 h. The reaction was quenched with dichloromethane and the insolubles were removed via filtration through a pad of celite. Concentration of the filtrate thus obtained afforded the title compound.

### Step 4: tert-Butyl trans-3-({cyclopropyl[3-(2-methoxyethoxy)-5-(3-methoxypropyl)benzyl]amino}carbonyl)-4-{1-[2-(2,6-dichloro-4-methylphenoxy)ethyl]-2-oxo-1,2-dihydro-4-pyridinyl}-4-hydroxy-1-piperidinecarboxylate

To a methanol solution (0.07 M) of tert-butyl trans-3-({cyclopropyl[3-(2-methoxyethoxy)-5-(3-methoxypropyl)benzyl]amino}carbonyl)-4-hydroxy-4-(2-oxo-1,2-dihydro-4-pyridinyl)-1-piperidinecarboxylate (1 eq.) from the previous step was added NaOH (2 N aq. solution, 3 eq.) and Mesylate 1 (3 eq.). The resulting mixture was then stirred at 60° C. for 16 h. The volatiles were then removed in vacuo and the residue thus obtained was directly subjected to purification by way of preparatory HPLC-MS (C-18 reverse phase column, 15 mL/min, 70:30 (v/v) H₂O:CH₃CN→5:95 (v/v) H₂O:CH₃CN).

### Step 5: trans-N-Cyclopropyl-4-{1-[2-(2,6-dichloro-4-methylphenoxy)ethyl]-2-oxo-1,2-dihydro-4-pyridinyl}-4-hydroxy-N-[3-(2-methoxyethoxy)-5-(3-methoxypropyl)benzyl]-3-piperidinecarboxamide

To a CH₂Cl₂ solution (0.05 M) of tert-butyl trans-3-({cyclopropyl[3-(2-methoxyethoxy)-5-(3-methoxypropyl)benzyl]amino}carbonyl)-4-{1-[2-(2,6-dichloro-4-methylphenoxy)ethyl]-2-oxo-1,2-dihydro-4-pyridinyl}4-hydroxy-1-piperidinecarboxylate (1 eq.) from the previous step was added HCl (4.0 M dioxane solution, 30 eq.). The resulting solution was stirred at RT for 3 h. Following the removal of the volatiles in vacuo, the resulting residue was directly loaded onto a SiO₂ column packed with 94:6 (v/v) CH₂Cl₂: 2.0 M NH₃ in MeOH. Elution with the same solvent system furnished the title compound. MS (ESI+, M+H): 716. ¹H NMR (acetone-d₆): δ (ppm) 0.75-1.04 (m, 4H), 1.74-1.83 (m, 3H), 2.29 (s, 3H), 2.57 (t, d=7.5 Hz, 2H), 2.66-2.73 (m, 1H), 2.85-2.87 (br m, 2H), 3.13-3.18 (br m, 3H), 3.28 (s, 3H), 3.32-3.36 (br m, 3H), 3.39 (s, 3H), 3.69 (t, d=6.8 Hz, 2H), 3.84-3.90 (m, 1H), 4.08 (t, d=6.8 Hz, 2H), 4.22-4.34 (m, 4H), 4.34 (d, J=13.4 Hz, 1H), 4.56 (d, J=13.4 Hz, 1H), 6.37-6.39 (br m, 2H), 6.51-6.58 (m, 3H), 6.63 (s, 1H), 7.22 (s, 2H), 7.58 (d, J=7.0 Hz, 1H).

### Example 2

### trans-N-Cyclopropyl-4-{1-[2-(2,6-dichloro-4-methylphenoxy)ethyl]-2-oxo-1,2-dihydro-4-pyridinyl)-4-methoxy-N-[3-(2-methoxyethoxy)-5-(3-methoxypropyl)benzyl]-3-piperidinecarboxamide

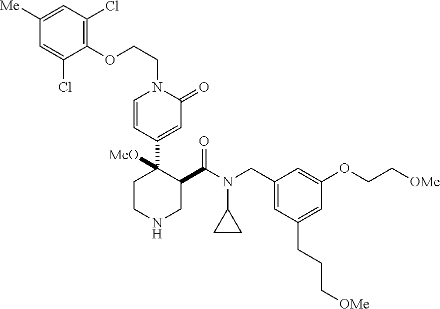

### Step 1: tert-Butyl trans-4-[2-(benzyloxy)-4-pyridinyl]-3-({cyclopropyl[3-(2-methoxyethoxy)-5-(3-methoxypropyl)benzyl]amino}carbonyl)-4-methoxy-1-1-piperidinecarboxylate

To a DMF solution (0.15 M) of tert-butyl trans-4-[2-(benzyloxy)-4-pyridinyl]-3-({cyclopropyl[3-(2-methoxyethoxy)-5-(3-methoxypropyl)benzyl]amino}carbonyl)-4-hydroxy-1-piperidinecarboxylate (1 eq., Example 1, Step 2) was added sodium hydride (1.2 eq.) and iodomethane (1.2 eq.). The reaction mixture was stirred at RT for 30 min before it was diluted with ether and water. The organic layer was separated and washed further with water and brine, dried over MgSO₄, filtered and the filtrate concentrated in vacuo. Purification of the crude product thus obtained by way of column chromatography (SiO₂, 3:2 (v/v) Hex: EtOAc→EtOAc) afforded the title compound.

### Step 2: tert-Butyl trans-3-({cyclopropyl[3-(2-methoxyethoxy)-5-(3-methoxypropyl)benzyl]amino}carbonyl)-4-methoxy-4-(2-oxo-1,2-dihydro-4-pyridinyl)-1-piperidinecarboxylate

To a solution of tert-butyl trans-4-[2-(benzyloxy)-4-pyridinyl]-3-({cyclopropyl[3-(2-methoxyethoxy)-5-(3-methoxypropyl)benzyl]amino}carbonyl)-4-methoxy-1-1-piperidinecarboxylate

(1 eq.) from the previous step in EtOAc (0.08 M) was added palladium (10% w/w on carbon, 0.5 eq.) and acetic acid (1.1 eq.). The resulting suspension was stirred under a balloon atmosphere of hydrogen for 4 h. The reaction was quenched with dichloromethane and the insolubles were removed via filtration through a pad of celite. Concentration of the filtrate thus obtained afforded the title compound.

### Step 3: tert-Butyl trans-3-({cyclopropyl[3-(2-methoxyethoxy)-5-(3-methoxypropyl)benzyl]amino}carbonyl)-4-{1-[2-(2,6-dichloro-4-methylphenoxy)ethyl]-2-oxo-1,2-dihydro-4-pyridinyl}-4-methoxy-1-piperidinecarboxylate

To a methanol solution (0.1 M) of tert-butyl trans-3-({cyclopropyl[3-(2-methoxyethoxy)-5-(3-methoxypropyl)benzyl]amino}carbonyl)-4-methoxy-4-(2-oxo-1,2-dihydro-4-pyridinyl)-1-piperidinecarboxylate (1 eq.) from the previous step was added NaOH (2 N aq. solution, 3 eq.) and Mesylate 1 (3 eq.). The resulting mixture was then stirred at 60° C. for 16 h. The volatiles were then removed in vacuo and the residue thus obtained was directly subjected to purification by way of preparatory HPLC-MS (C-18 reverse phase column, 15 mL/min, 70:30 (v/v) H₂O:CH₃CN→5:95 (v/v) H₂O:CH₃CN).

### Step 4: trans-N-Cyclopropyl-4-{1-[2-(2,6-dichloro-4-methylphenoxy)ethyl]-2-oxo-1,2-dihydro-4-pyridinyl}-4-methoxy-N-[3-(2-methoxyethoxy)-5-(3-methoxypropyl)benzyl]-3-piperidinecarboxamide

To a CH₂Cl₂ solution (0.06 M) of tert-butyl trans-3-({cyclopropyl[3-(2-methoxyethoxy)-5-(3-methoxypropyl)benzyl]amino}carbonyl)-4-{1-[2-(2,6-dichloro-4-methylphenoxy)ethyl]-2-oxo-1,2-dihydro-4-pyridinyl}4-methoxy-1-piperidinecarboxylate (1 eq.) from the previous step was added HCl (4.0 M dioxane solution, 30 eq.). The resulting solution was stirred at RT for 3 h. Following the removal of the volatiles in vacuo, the resulting residue was directly loaded onto a SiO₂ column packed with 94:6 (v/v) CH₂Cl₂: 2.0 M NH₃ in MeOH. Elution with the same solvent system furnished the title compound. MS (ESI+, M+H): 730. ¹H NMR (acetone-d₆): δ (ppm) 0.75-1.03 (m, 4H), 1.78-1.84 (m, 2H), 2.26-2.47 (m, 5H), 2.52-2.64 (m, 3H), 2.72 (br s, 1H), 3.02 (s, 3H), 3.11-3.23 (br m, 4H), 3.26 (s, 3H), 3.33-3.35 (m, 5H), 3.68 (t, d=6.9 Hz, 2H), 3.92 (br s, 1H), 4.13 (t, d=6.9 Hz, 2H), 4.19-4.39 (m, 5H), 4.93 (br d, J=13.0 Hz, 1H), 6.39 (d, J=6.8 Hz, 1H), 6.53 (s, 1H), 6.69 (s, 1H), 6.74 (s, 1H), 6.76 (s, 1H), 7.22 (s, 2H), 7.59 (d, J=6.8 Hz, 1H).

### Example 3

### trans-N-Cyclopropyl-4-{1-[3-(2,6-dichloro-4-methylphenoxy)propyl]-2-oxo-1,2-dihydro-4-pyridinyl}-4-hydroxy-N-[3-(2-methoxyethoxy)-5-(3-methoxypropyl)benzyl]-3-piperidinecarboxamide

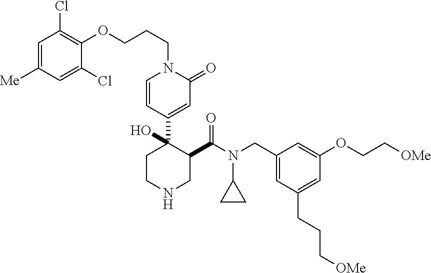

Prepared according to the procedure described in Example 1 but using instead Mesylate 2 as alkylation reagent in Step 4. MS (ESI+, M+H): 730.

### Example 4

### trans-N-Cyclopropyl-4-{1-[3-(2,6-dichloro-4-methylphenoxy)propyl]-2-oxo-1,2-dihydro-4-pyridinyl)-4-methoxy-N-[3-(2-methoxyethoxy)-5-(3-methoxypropyl)benzyl]-3-piperidinecarboxamide

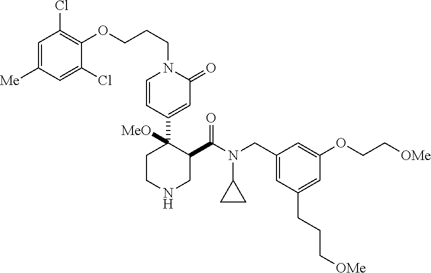

Prepared according to the procedure described in Example 2 but using instead Mesylate 2 as alkylation reagent in Step 3. MS (ESI+, M+H): 744.

### Example 5

### trans-N-[2-Chloro-5-(2-methoxyethyl)benzyl]-N-cyclopropyl-4-{1-[3-(2,6-dichloro-4-methylphenoxy)propyl]-2-oxo-1,2-dihydro-4-pyridinyl)-4-hydroxy-3-piperidinecarboxamide

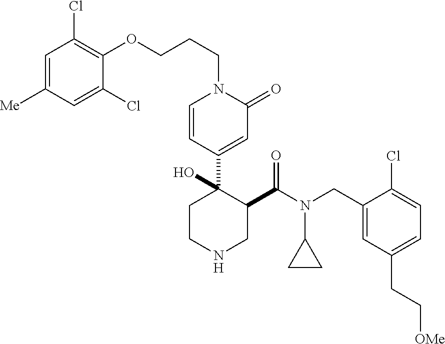

Prepared according to the procedure described in Example 1 but using instead Amine 2 as starting material in Step 1 and Mesylate 2 as alkylation reagent in Step 4. MS (ESI+, M+H): 676. ¹H NMR (acetone-d₆): δ (ppm) 0.75-1.02 (m, 4H), 1.48-1.52 (br m, 1H), 1.79-1.84 (m,

1H), 2.23-2.28 (m, 3H), 2.31 (s, 3H), 2.72 (t, d=7.3 Hz, 2H), 2.75-2.90 (m, 5H), 3.14-3.28 (m, 7H), 3.51 (t, d=7.3 Hz, 2H), 3.92-4.28 (m, 5H), 4.49 (d, J=13.1 Hz, 1H), 4.55 (d, J=13.1 Hz, 1H), 6.42 (d, J=6.8 Hz, 1H), 6.53 (s, 1H), 6.72 (s, 1H), 7.11 (d, J=6.8 Hz, 1H), 7.25-7.27 (m, 3H), 7.58 (d, J=7.0 Hz, 1H).

1-20. (canceled)

21. A compound of formula I, 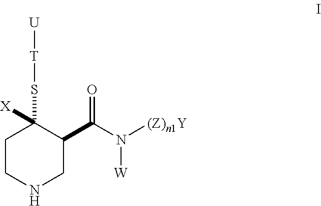
or a pharmaceutically acceptable salt thereof,
wherein:
S is 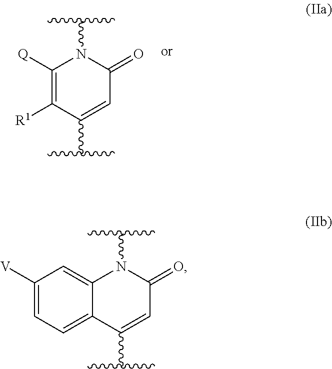
wherein the nitrogen atom in IIa and IIb above is attached to T;
T is a bond; —(CH₂)ᵣ; —(CH₂)ᵣ-A-(CH₂)ₛ—; or —(CH₂)ᵣ-A-(CH₂)ᵣ—B—;
A and B are independently selected from the group consisting of —O—, —S—, —S(O)— and —S(O)₂—;
r is the integer 2, 3, 4, or 5;
s is the integer 0, 1, or 2;
U is unsubstituted aryl; mono-, di-, tri- or tetra-substituted aryl wherein the substituents are independently selected from the group consisting of halogen, alkyl, alkoxy, cyano and —CF₃; or mono-, di-, or tri-substituted heteroaryl wherein the substituents are independently selected from the group consisting of halogen, alkyl, alkoxy, cyano and —CF₃;
V is selected from the group consisting of: hydrogen, halogen, C₁-C₆ alkyl, C₃-C₆ cycloalkyl, C₂-C₆ alkenyl, C₃-C₆ cycloalkenyl, C₂-C₆ alkynyl, cyano and C₁-C₅ alkoxy,
wherein said alkyl, cycloalkyl, alkenyl, cycloalkenyl, alkynyl and alkoxy are optionally substituted with 1-3 substituents, each of which is independently selected from the group consisting of: halogen, C₁-C₅ alkyl, C₂-C₅ alkenyl, cyano and C₁-C₅ alkoxy, wherein each of the foregoing alkyl, alkenyl and alkoxy substituents is optionally substituted with 1-3 halogens;
Q and R¹ are independently selected from the group consisting of: hydrogen, halogen, C₁-C₅ alkyl, C₃-C₈ cycloalkyl, C₂-C₅ alkenyl, C₃-C₈ cycloalkenyl, C₂-C₅ alkynyl, cyano and C₁-C₅ alkoxy, aryl and heteroaryl;
wherein said aryl and heteroaryl are optionally substituted with 1-4 halogens,
wherein said alkyl, cycloalkyl, alkenyl, cycloalkenyl, alkynyl and alkoxy are optionally substituted with 1-3 substituents, each of which is independently selected from the group consisting of: halogen, C₁-C₅ alkyl, C₂-C₅ alkenyl, cyano and C₁-C₅ alkoxy, wherein each of the foregoing alkyl, alkenyl and alkoxy substituents is optionally substituted with 1-3 halogens;
W is cyclopropyl, unsubstituted or mono-, di-, tri-, tetra- or penta-substituted with halogen;
X is selected from the group consisting of: OR², R², —(C₁-C₅ alkylene)-(O)₀₋₁-aryl and —(C₁-C₅ alkylene)-(O)₀₋₁-heteroaryl,
wherein R² is selected from the group consisting of: hydrogen, C₁-C₅ alkyl, C₃-C₈ cycloalkyl, C₂-C₅ alkenyl, C₃-C₈ cycloalkenyl, C₂-C₅ alkynyl, C₁-C₅-cyano, —(C₁-C₅ alkylene)-O—R³, —(C₁-C₅ alkylene)-N(—R³)—C(═O)—(C₁-C₅ alkyl), —(C₁-C₅ alkylene)-C(═O)—N(—R³)—(C₁-C₅ alkyl), —(C₁-C₅ alkylene)-N(—R³)—C(═O)—O—(C₁-C₅ alkyl), —(C₁-C₅ alkylene)-O—C(═O)—N(—R³)—(C₁-C₅ alkyl), —(C₁-C₅ alkylene)-N(—R³)—(C₁-C₅ alkyl), —(C₁-C₅ alkylene)-S—(C₁-C₅ alkyl), —(C₁-C₅ alkylene)-S(═O)—(C₁-C₅ alkyl) and —(C₁-C₅ alkylene)-S(═O)₂—(C₁-C₅ alkyl),
wherein R², except hydrogen, is optionally substituted with 1-3 substituents, independently selected from the group consisting of: halogen, C(═O)OH, C₁-C₅ alkyl, C₂-C₅ alkenyl, and C₁-C₅ alkoxy, wherein each of the alkyl, alkenyl, and alkoxy substituents is optionally substituted with 1-3 halogens,
wherein the heteroaryl of the —(C₁-C₅ alkylene)-(O)₀₋₁-heteroaryl contains 1-3 heteroatoms, independently selected from the group consisting of: N, O and S, wherein each N is optionally in the form of an oxide and each S is optionally in the form of an oxide selected from the group consisting of: S(═O) and S(═O)₂,
wherein the aryl and heteroaryl of —(C₁-C₅ alkylene)-(O)₀₋₁-aryl and —(C₁-C₅ alkylene)-(O)₀₋₁-heteroaryl, respectively, are optionally substituted with 1-4 halogens, and
wherein R³ is selected from the group consisting of: hydrogen, C₁-C₆ alkyl, C₃-C₆ cycloalkyl, C₂-C₆ alkenyl, C₃-C₆ cycloalkenyl, and C₂-C₆ alkynyl, wherein each of the foregoing alkyl, cycloalkyl, alkenyl, cycloalkenyl and alkynyl substituents is optionally substituted with 1-3 halogens;
Z is C₁-C₂ alkylene optionally substituted with 1-2 substituents, independently selected from the group consisting of: halogen, C₁-C₃ alkyl and C₃ cycloalkyl, wherein the foregoing alkyl and cycloalkyl substituents are optionally substituted with 1-3 halogens;
n1 is 0 or 1;
Y is (i) a five- or six-membered saturated or unsaturated heterocyclic or carbocyclic monocyclic ring (“monocyclic ring”) or (ii) a fused ring system which is a five- or six-membered saturated or unsaturated heterocyclic or carbocyclic ring which is fused to a five- or six-membered saturated or unsaturated heterocyclic or carbocyclic ring (“fused ring”),
wherein the heterocyclic ring(s) of (i) or (ii) contain from 1-3 heteroatoms, independently selected from N, O and S, wherein each N is optionally in the form of an oxide and each S is optionally in the form of an oxide selected from the group consisting of: S(═O) and S(═O)₂,
wherein the heterocyclic or carbocyclic ring(s) of (i) or (ii) is optionally mono-, di-, tri-, tetra-, penta- or hexa-substituted, each substituent of which is independently selected from the group consisting of:
(1) halogen,
(2) —OH,
(3) —NH(R⁴),
(4) oxo,
(5) —C(═O)—R⁴,
(6) —O—C(═O)—R⁴,
(7) C₁-C₅ alkyl optionally substituted with 1-3 halogens,
(8) C₃-C₈ cycloalkyl optionally substituted with 1-3 halogens,
(9) C₂-C₅ alkenyl optionally substituted with 1-3 halogens,
(10) C₃-C₈ cycloalkenyl optionally substituted with 1-3 halogens,
(11) C₂-C₅ alkynyl optionally substituted with 1-3 halogens,
(12) C₁-C₅ alkoxy optionally substituted with 1-3 halogens,
(13) cyano,
(14) C₁-C₅-cyano optionally substituted with 1-3 halogens,
(15) —OCF₃,
(16) —C(R⁵)₃,
(17) —(C₁-C₅ alkylene)-OR⁶ optionally substituted with 1-3 halogens,
(18) —N(R⁴)—(C₁-C₅ alkylene)-OR⁶ optionally substituted with 1-3 halogens,
(19) —O—(C₁-C₅ alkylene)-OR⁶ optionally substituted with 1-3 halogens,
(20) —S—(C₁-C₅ alkylene)-OR⁶ optionally substituted with 1-3 halogens,
(21) —S(═O)—(C₁-C₅ alkylene)-OR⁶ optionally substituted with 1-3 halogens,
(22) —S(═O)₂—(C₁-C₅ alkylene)-OR⁶ optionally substituted with 1-3 halogens,
(23) —(C₁-C₅ alkylene)-N(R⁴)—C(═O)—(C₁-C₅ alkylene)-R⁶ optionally substituted with 1-3 halogens,
(24) —(C₁-C₅ alkylene)-N(R⁴)—C(═O)—OR⁶ optionally substituted with 1-3 halogens,
(25) —(C₁-C₅ alkylene)-N(R⁴)(R⁶) optionally substituted with 1-3 halogens,
(26) —O—(C₁-C₅ alkylene)-C(R⁴)₂—C(═O)OR⁶ optionally substituted with 1-3 halogens,
(27) —(C₁-C₅ alkylene)-C(R⁴)₂—C(═O)—OR⁶ optionally substituted with 1-3 halogens,
(28) —O—(C₁-C₅ alkylene)-morpholine optionally substituted with 1-3 halogens,
(29) —OC(═O)-morpholine,
(30) —SR⁶,
(31) —S(═O)—R⁶,
(32) —S(═O)₂—R⁶
(33) —N(R⁴)(R⁶),
(34) —(C₁-C₅ alkylene)-C(R⁴)₂—(R⁶) optionally substituted with 1-3 halogens,
(35) —(R⁷)₀₋₁R⁸,
(36) C₂-C₅ alkenyl-OR⁶ optionally substituted with 1-3 halogens,
(37) C₂-C₅ alkynyl-OR⁶ optionally substituted with 1-3 halogens,
(38) —(C₁-C₅ alkylene)-C(═O)—(C₁-C₅ alkylene)-R⁶ optionally substituted with 1-3 halogens,
(39) —(C₁-C₅ alkylene)-O—C(═O)—(C₁-C₅ alkylene)-R⁶ optionally substituted with 1-3 halogens,
(40) —(C₁-C₅ alkylene)-C(═O)—N(R⁴)(R⁶) optionally substituted with 1-3 halogens,
(41) —(C₁-C₅ alkylene)-O—C(═O)—N(R⁴)(R⁶) optionally substituted with 1-3 halogens,
(42) —(C₁-C₅ alkylene)-SR⁶ optionally substituted with 1-3 halogens,
(43) —(C₁-C₅ alkylene)-S(═O)—R⁶ optionally substituted with 1-3 halogens, and
(44) —(C₁-C₅ alkylene)-S(═O)₂—R⁶ optionally substituted with 1-3 halogens,
wherein R⁴ is selected from the group consisting of: hydrogen, C₁-C₆ alkyl, C₃-C₈ cycloalkyl, C₂-C₆ alkenyl, C₃-C₈ cycloakenyl and C₂-C₆ alkynyl, wherein each of the foregoing alkyl, cycloalkyl, alkenyl, cycloalkenyl and alkynyl substituents is optionally substituted with 1-3 halogens,
wherein R⁵ is halogen,
wherein R⁶ is selected from the group consisting of: hydrogen, C₁-C₆ alkyl, C₃-C₈ cycloalkyl, C₂-C₆ alkenyl, C₃-C₈ cycloalkenyl and C₂-C₆ alkynyl, wherein each of the foregoing alkyl, cycloalkyl, alkenyl, cycloalkenyl and alkynl substituents is optionally substituted with 1-3 halogens,
wherein R⁷ is selected from the group consisting of: —C(H)(OH)—, —C(═O)—, —OC(═O)—, —C(═O)O—, —O—, —OC(═O)O—, C₁-C₅ alkylene, C₂-C₅ alkenylene, —N(R⁴)—, —S—, —S(═O)—, —S(═O)₂—, —N(R⁴)—C(═O)—, —C(═O)—N(R⁴)—, —OC(═O)—N(R⁴)—, —N(R⁴)—C(═O)O—, —N(R⁴)—S(═O)₂—, and —S(═O)₂—N(R⁴)—, wherein each of the foregoing alkylene and alkenylene substituents is optionally substituted with 1-3 halogens, and wherein R⁴ is defined above, and
wherein R⁸ is a five- or six-membered saturated or unsaturated heterocyclic or carbocyclic ring which is optionally mono-, di-, tri-, tetra- or penta-substituted, wherein each substituent is independently selected from the group consisting of: halogen, —OH, —SR⁴, —N(R⁴)(R⁶), C₁-C₅ alkyl, C₃-C₈ cycloalkyl, C₂-C₅ alkenyl, C₃-C₆ cycloalkenyl, C₂-C₅ alkynyl, C₁-C₅ alkoxy, cyano and C₁-C₅-cyano, wherein said heterocyclic ring contains from 1 to 3 heteroatoms, independently selected from N, O and S, wherein each N is optionally in the form of an oxide and each S is optionally is in the form of an oxide selected from the group consisting of: S(═O)and S(═O)₂, and wherein R⁴ and R⁶ are defined above.

22. The compound of claim 21 wherein S is: 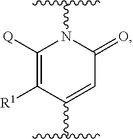
wherein the nitrogen atom in S above is attached to T; and
wherein Q and R₁ are defined in claim 21,
or a pharmaceutically acceptable salt thereof.

23. The compound of claim 21 wherein Y is: 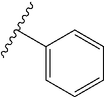
optionally mono-, di-, tri-, tetra- or penta-substituted as described in claim 21,
or a pharmaceutically acceptable salt thereof.

24. The compound of claim 21 wherein S is: 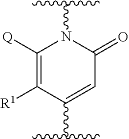
wherein the nitrogen atom in S above is attached to T;
wherein Q and R¹ are as defined in claim 21, and
wherein Y is: 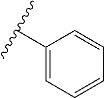
optionally mono-, di-, tri-, tetra- or penta-substituted as described in claim 21,
or a pharmaceutically acceptable salt thereof.

25. The compound of claim 21 wherein T is —CH₂—CH₂—O— or —CH₂—CH₂—CH₂—O—,
or a pharmaceutically acceptable salt thereof.

26. The compound of claim 21 wherein U is a mono, di-, tri-or tetra-substituted aryl,
or a pharmaceutically acceptable salt thereof.

27. The compound of claim 21 wherein U is a mono, di-, tri-or tetra-substituted phenyl wherein the substituents are independently selected from the group consisting of, halogen, C₁-C₆ alkyl, C₁-C₆ alkoxy, cyano and —CF₃,
or a pharmaceutically acceptable salt thereof.

28. The compound of claim 21 wherein U is 2,6-dichloro-4methyl-phenyl,
or a pharmaceutically acceptable salt thereof.

29. The compound of claim 21 wherein Q and R¹ are independently selected from the group consisting of: H, —OCH₂OCH₃— and —CH₃,
or a pharmaceutically acceptable salt thereof.

30. The compound of claim 21 wherein V is hydrogen or halogen,
or a pharmaceutically acceptable salt thereof.

31. The compound of claim 21 wherein W is unsubstituted cyclopropyl,
or a pharmaceutically acceptable salt thereof.

32. The compound of claim 21 wherein X is —H, —OH, or —OCH₃,
or a pharmaceutically acceptable salt thereof.

33. The compound of claim 21 wherein (Z)ₙ₁ is —CH₂—,
or a pharmaceutically acceptable salt thereof.

34. The compound of claim 21 wherein:
S is 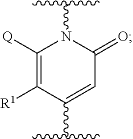
wherein the nitrogen atom in S above is attached to T,
U is an optionally mono-, di-, tri-, or tetra-substituted phenyl ring, wherein substituents are independently selected from the group consisting of: halogen, alkyl, alkoxy, cyano and —CF₃,
W is cyclopropyl, unsubstituted or mono-, di-, tri-, tetra- or penta-substituted with halogen;
X is hydrogen, OH or methoxy,
(Z)ₙ₁ is —CH₂—,
Y is 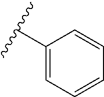
optionally mono-, di-, tri-, tetra- or penta-substituted as described in claim 21,
or a pharmaceutically acceptable salt thereof.

35. The compound of claim 21 wherein:
S is 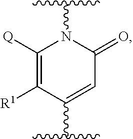
wherein the nitrogen atom in S above is attached to T,
T is —(CH₂)ᵣ—O—,
U is an optionally mono-, di-, tri-, or tetra-substituted phenyl ring, wherein substituents are independently selected from the group consisting of: halogen, alkyl, alkoxy, cyano and —CF₃,
W is cyclopropyl, unsubstituted or mono-, di-, tri-, tetra- or penta-substituted with halogen;
X is hydrogen, OH or methoxy,
(Z)ₙ₁ is —CH₂—,
Y is 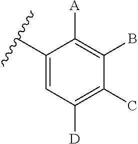
wherein:
A is selected from the group consisting of:
(1) hydrogen,
(2) halogen,
(3) C₁-C₅ alkyl,
(4) C₁-C₅ alkoxy, and
(5) —S—(CH₂)₀₋₃—CH₃,
wherein (3) and (4) are optionally substituted with 1-3 halogens,
B is selected from the group consisting of:
(1) hydrogen,
(2) halogen,
(3) C₁-C₅ alkyl,
(4) C₁-C₅ alkoxy,
(5) —OH,
(6) —CF₃,
(7) —C(═O)—CH₃,
(8) —O—(C₁-C₅ alkylene)-O-cyclopropyl,
(9) —O—(C₁-C₅ alkylene)-O—(CH₂)₀₋₂—CH₃,
(10) —(C₁-C₅ alkylene)-O—(CH₂)₀₋₂—CH₃,
(11) —OC(═O)-morpholine,
(12) —O—(C₁-C₅ alkylene)-morpholine,
(13) —O—(C₁-C₅ alkylene)-C(CH₃)₂—C(═O)OH,
(14) —O—(C₁-C₅ alkylene)-C(CH₃)₂—C(═O)OCH₃, 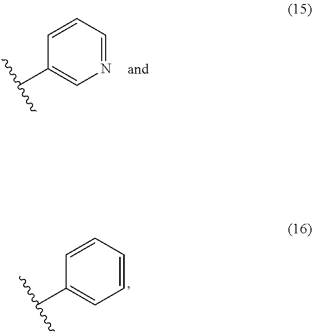
wherein (3), (4), (8), (9), (10), (12), (13), (14), (15) and (16) are optionally substituted with 1-3 halogens,
C is selected from the group consisting of:
(1) hydrogen,
(2) halogen,
(3) C₁-C₅ alkyl optionally substituted with 1-3 halogens, and
(4) C₁-C₅ alkoxy optionally substituted with 1-3 halogens, and
D is selected from the group consisting of:
(1) hydrogen,
(2) halogen,
(3) C₁-C₅ alkyl,
(4) C₁-C₅ alkoxy,
(5) C₁-C₅-cyano,
(6) C₂-C₅ alkenylene-O—(CH₂)₀₋₂—CH₃,
(7) —(C₁-C₅ alkylene)-N(H)—C(═O)—O—(CH₂)₀₋₂—CH₃,
(8) —(C₁-C₅ alkylene)-N(H)—C(═O)—(CH₂)₀₋₂—CH₃,
(9) —(C₁-C₅ alkylene)-O—CHF₂,
(10) —(C₁-C₅ alkylene)-O—(CH₂)₀₋₂—CH₃,
(11) —O—(C₁-C₅ alkylene)-O—(CH₂)₀₋₂—CH₃,
(12) —(C₁-C₅ alkylene)-OH,
(13) —S—(C₁-C₅ alkylene)-OH,
(14) —SCF₃
(15) —N(H)—(C₁-C₅ alkylene)-O—(CH₂)₀₋₂—CH₃, and
(16) 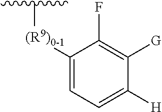
wherein F, G and H are independently selected from the group consisting of: hydrogen, halogen and C₁-C₃ alkyl optionally substituted with 1-3 halogens, and
wherein R⁹ is selected from the group consisting of: —CH₂—, —C(H)(OH)— and —C(═O)—,
wherein (3), (4), (5), (6), (7), (8), (9), (10), (11), (12), (13) and (15) are optionally substituted with 1-3 halogens,
or a pharmaceutically acceptable salt thereof.

36. The compound of claim 21 wherein:
S is 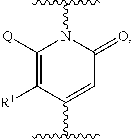
wherein the nitrogen atom in S above is attached to T,
T is —CH₂—CH₂—O— or —CH₂—CH₂—CH₂—O—,
U is 2,6-dichloro-4-methyl-phenyl,
Q and R¹ are independently selected from the group consisting of: —H, —OCH₂OCH₃— and —CH₃,
W is unsubstituted cyclopropyl,
X is hydrogen, OH or methoxy,
(Z)ₙ₁ is —CH₂—,
Y is 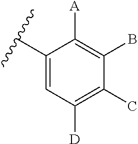
wherein:
A is selected from the group consisting of: hydrogen, halogen, C₁-C₅ alkyl and C₁-C₅ alkoxy,
B is selected from the group consisting of: hydrogen, halogen, C₁-C₅ alkyl, C₁-C₅ alkoxy and —O—(C₁-C₅ alkylene)-O—(CH₂)₀₋₂—CH₃,
C is selected from the group consisting of: hydrogen, halogen, C₁-C₅ alkyl and C₁-C₅ alkoxy,
D is selected from the group consisting of: hydrogen, halogen, C₁-C₅ alkyl, C₁-C₅ alkoxy and —(C₁-C₅ alkylene)-O—(CH₂)₀₋₂—CH₃,
wherein the alkyl or alkoxy groups of A-D are optionally substituted with 1-3 halogens,
or a pharmaceutically acceptable salt thereof.

37. The compound of claim 21 which is selected from the group consisting of: 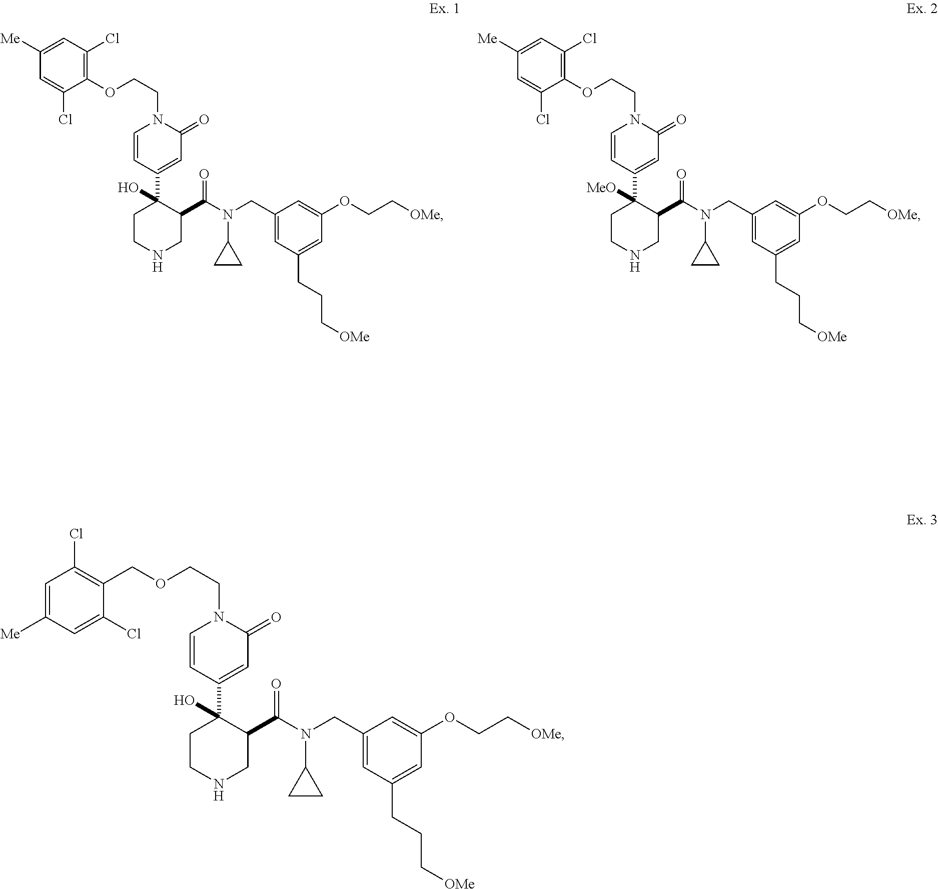 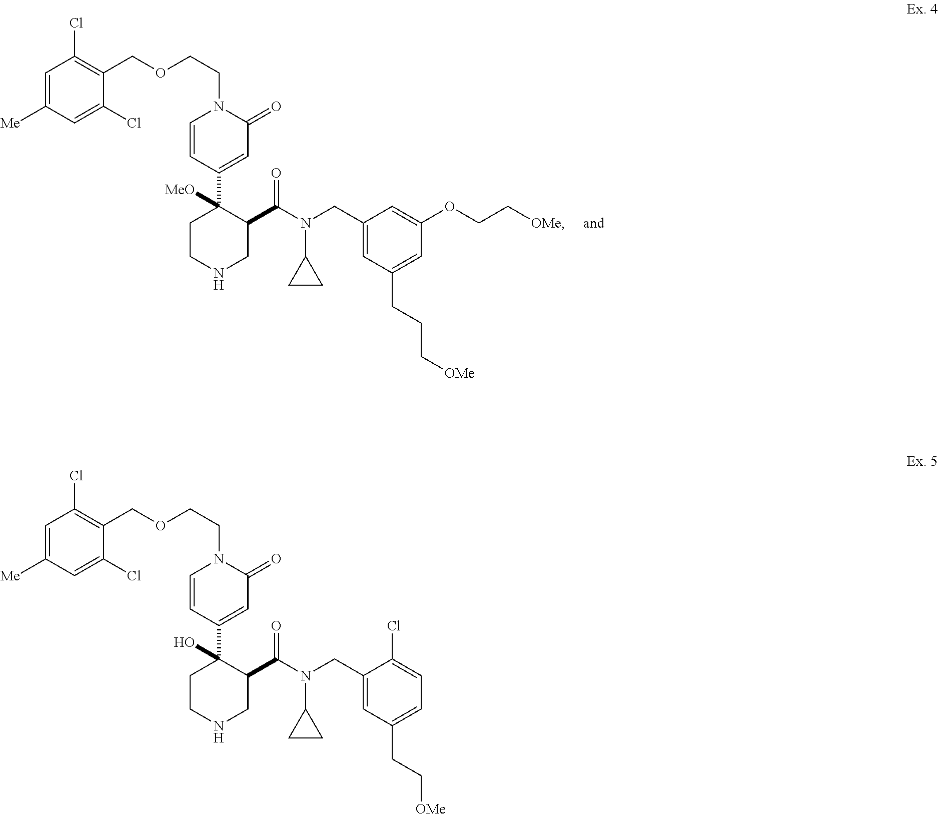
or a pharmaceutically acceptable salt thereof.

38. A pharmaceutical composition comprising an effective amount of a compound according to claim 21 or a pharmaceutically acceptable salt thereof, and a pharmaceutically acceptable carrier.

39. Use of a compound according to claim 21, or a pharmaceutically acceptable salt thereof, for the manufacture of a medicament for the treatment of a disease related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, or other diseases known to be related to the renin-angiotensin system.

40. A method for the treatment of a disease related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, or other diseases known to be related to the renin-angiotensin system, comprising the administration to a patient of a pharmaceutically active amount of a compound according to claim 21, or a pharmaceutically acceptable salt thereof.
